(19) United States
(12) Patent Application Publication
(10) Pub. No.: US 2011/0054359 A1
(43) Pub. Date: Mar. 3, 2011

(54) Footwear-based body weight monitor and postural allocation, physical activity classification, and energy expenditure calculator

(75) Inventors: Eduard Sazonov, Potsdam, NY (US); Raymond Browning, Fort Collins, CO (US); James Hill, Centennial, CO (US); Yves Schutz, Vevey (CH)

(73) Assignee: The Regents of the University of Colorado , a body corporate, Denver, CO (US)

(21) Appl. No.: 12/709,131

(22) Filed: Feb. 19, 2010

Related U.S. Application Data
(60) Provisional application No. 61/208,196, filed on Feb. 20, 2009.
(60) Provisional application No. 61/256,132, filed on Oct. 29, 2009.
(60) Provisional application No. 61/266,319, filed on Dec. 3, 2009.

(51) Int. Cl. A61B 5/11

(52) U.S. Cl. 600/595

(57) ABSTRACT

A footwear system for monitoring weight, posture allocation, physical activity classification, and energy expenditure calculation includes an accelerometer configured to obtain acceleration data indicative of movement of a user's foot or leg. The footwear system may also include a pressure sensing device mounted in an insole and configured to obtain pressure data indicative of pressure applied by a user's foot to the insole, as well as a transmitter communicatively coupled to both the accelerometer and the pressure sensing device and configured to transmit the acceleration and pressure data to a first processing device configured process the acceleration data and the pressure data to distinguish a first posture from a second posture different from the first posture and process the acceleration data and the pressure data to distinguish a first movement-based activity from a second movement-based activity different from the first movement-based activity. The footwear system may also include a second processing device communicatively coupled to the first processing device and configured to derive a second energy expenditure value.

### CROSS REFERENCE TO RELATED APPLICATIONS

The present application claims priority to U.S. Provisional Patent Application No. 61/208,196, filed 20 Feb. 2009, entitled, “Footwear-based System and Method for Monitoring Body Weight, Postural Allocation, and Energy Expenditure,” U.S. Provisional Patent Application No. 61/256,132 filed 29 Oct. 2009, entitled “Shoe-based Wearable Sensor for Monitoring Body Weight, Postural Allocation, and Energy Expenditure,” and U.S. Provisional Patent Application No. 61/266,319, filed 3 Dec. 2009, entitled “A novel wireless, wearable shoe-based system for weight and physical activity management”, each of which is incorporated by reference herein in its entirety.

### STATEMENT REGARDING FEDERALLY SPONSORED RESEARCH OR DEVELOPMENT

This technology was developed in part with sponsorship by National Institutes of Health Grant No. 1R43DK083229-01A1 and the U.S. federal government may have certain rights to this technology.

### TECHNICAL FIELD

The invention relates generally to weight management devices, and more particularly to a footwear-based system for monitoring body weight, postural allocation, physical activity classification, and energy expenditure calculation, and providing feedback aimed at maintaining healthy levels of physical activity and weight management.

### BACKGROUND

Many Americans and adults worldwide are overweight or obese. Obesity is due to a sustained positive energy balance, i.e., in which an individual's energy intake is greater than the individual's energy expenditure. Weight gain is typically coupled with low levels of physical activity and sedentary lifestyles. As a result, most weight management programs recommend regular monitoring of body weight and increased energy expenditure lifestyle alterations that increase physical activity levels.

While increasing energy expenditure can be achieved via exercise, there are other components of daily energy expenditure, such as non-exercise activity thermogenesis (NEAT), which also play an important role in weight management. NEAT includes the energy expenditure associated with posture allocation, for example, time spent lying, sitting and standing, as well as energy expenditure associated with non-exercise movement, for example, walking and other free-living movements. In fact, the energy expended each day via non-exercise movement and posture can be greater than that of a vigorous exercise session. Because the positive daily energy balance associated with weight gain may be relatively small, i.e., on the order of 25-100 kcal/day, relatively minor adjustments in daily activity patterns may promote weight loss and prevent weight gain.

While body weight can be monitored using an electronic scale, scales are not discreet and may not be used throughout the day by individuals who are sensitive about their weight. Devices that quantitatively monitor levels of physical activity, e.g., accelerometers have been shown to improve weight management outcomes. These devices are also able to more accurately estimate activity energy expenditure, as they can distinguish activities from stationary postures, which have different metabolic rates. However, current devices are incapable of differentiating between different postures, e.g., sitting and standing, and instead group these postures together. These devices further cannot differentiate between different activities, for example, cycling and ascending and descending stairs.

The information included in this Background section of the specification, including any references cited herein and any description or discussion thereof, is included for technical reference purposes only and is not to be regarded subject matter by which the scope of the invention is to be bound.

### SUMMARY

The wearable energy expenditure monitoring system disclosed herein assists users in losing weight and maintaining healthy level of physical activity by calculating body weight, allocating posture, classifying physical activity, and calculating energy expended by a user and providing feedback to the user based on the calculated energy expenditure. In one embodiment, the monitoring system may calculate the energy expended by the user based on a combination of acceleration data collected from an accelerometer and pressure data collected from a pressure sensor. The acceleration and pressure data may be transmitted to a processing device, which may provide periodic feedback to the user regarding his or her calculated energy expenditure. Accordingly, the wearable monitoring system may assist individuals in achieving and maintaining a healthy body weight though monitoring of physical activity and encouraging health-promoting lifestyle changes.

One embodiment may take the form of a footwear system for monitoring weight, posture allocation, physical activity classification, and energy expenditure calculation includes an accelerometer configured to obtain acceleration data indicative of movement of a user's foot or leg. The footwear system may also include a pressure sensing device mounted in an insole and configured to obtain pressure data indicative of pressure applied by a user's foot to the insole, as well as a transmitter communicatively coupled to both the accelerometer and the pressure sensing device and configured to transmit the acceleration and pressure data to a first processing device configured process the acceleration data and the pressure data to distinguish a first posture from a second posture different from the first posture and process the acceleration data and the pressure data to distinguish a first movement-based activity from a second movement-based activity different from the first movement-based activity.

Another embodiment may take the form of a method executed by a processing device for recognizing posture and activities using the processing device. The method may include receiving pressure data indicative of pressure applied by a user's foot to the insole, receiving acceleration data from an accelerometer indicative of movement of a user's foot or leg, and processing the pressure and acceleration data so as to distinguish a first posture from a second posture and to distinguish a first movement-based activity from a second movement-based activity.

Yet another embodiment may take the form of a method for deriving an energy expenditure value. The method may include obtaining pressure data using a capacitive pressure sensor indicative of pressure applied by a user's foot to the insole. The capacitive pressure sensor may include one or more conducting plates. The method may also include obtaining acceleration data using an accelerometer indicative of movement of a user's foot or leg and transmitting the pressure and acceleration data to a processing device configured to process the pressure and acceleration data and derive an energy expenditure value based on the pressure and acceleration data.

Another embodiment may take the form of a computer-readable medium having computer-executable instructions for performing a computer process for recognizing posture and activities. The instructions include causing the computer server to receive pressure data indicative of pressure applied by a user's foot to an insole, receive acceleration data from an accelerometer indicative of movement of a user's foot or leg, and process the pressure and acceleration data so as to distinguish a first posture from a second posture and to distinguish a first movement-based activity from a second movement-based activity.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present disclosure is provided in the following written description of various embodiments, illustrated in the accompanying drawings, and defined in the appended claims.

### BRIEF DESCRIPTION OF THE DRAWINGS

FIG. 1A illustrates a first embodiment of a wearable energy expenditure monitoring system.

FIG. 1B illustrates a second embodiment of a wearable energy expenditure monitoring system.

FIG. 1C illustrates a schematic diagram of the embodiments of the wearable energy expenditure monitoring system shown in FIGS. 1A and 1B.

FIG. 2A illustrates a top view of an insole incorporating a capacitive pressure sensor.

FIG. 2B illustrates a cross-sectional view of a user's foot and the insole shown in FIG. 2A, as taken along line 2B-2B.

FIG. 2C illustrates a perspective view of the insole shown in FIG. 2A.

FIG. 2D illustrates a circuit diagram of the insole shown in FIG. 2A.

FIG. 3 illustrates an embodiment of a circuit that is configured to perform capacitive pressure sensing using the capacitive pressure sensor shown in FIGS. 2A-2D.

FIG. 4 illustrates a top view of an insole incorporating force sensitive resistor pressure sensors.

FIG. 5 is a schematic diagram illustrating information flow between a pressure sensor, an accelerometer, a physiological sensor and a processing device.

FIG. 6 illustrates a flow diagram of a method for monitoring energy expenditure and body weight using pressure and acceleration data.

FIGS. 7A-7B illustrate a flow diagram of a method for monitoring body weight and energy expenditure using pressure and acceleration data.

FIG. 8 is a bar graph illustrating 6-class average validation accuracy obtained by individual, and 16- and 9-subject group models for each subject.

FIG. 9 is a table depicting the backward selection of various sensor configurations.

FIG. 10 is a line graph of the posture/activity recognition accuracy of sensors as a function of the decimation factor.

FIGS. 11A-11E depict two-dimensional representations of feature vectors for each posture/activity from captured sensor data.

FIGS. 12A-12H depict Bland-Altman plots (constructed for both EE, kcal·min⁻¹ and EE, METs prediction) for four shoe-based models of energy expenditure.

FIGS. 13A-13H depict plots of Passing-Bablok regression analysis for four shoe-based models of energy expenditure.

### DETAILED DESCRIPTION

Embodiments disclosed herein include a wearable energy expenditure monitoring system for monitoring body weight, postural allocation, physical activity classification, and energy expenditure calculation. “Posture allocation,” as used herein, includes distinguishing between various postures that may be held by a user. Some examples of postures may include, but are not limited to, lying down, sitting, standing, and so on and so forth. “Physical activity classification,” as used herein, includes distinguishing between various movement-based activities performed by a user. For example, physical activities may include, but are not limited to, walking, jogging, running, cycling, climbing stairs, and so on and so forth.

In one embodiment, the wearable monitoring system may include an accelerometer and a pressure sensor that is integrated into an insole. An “insole,” as used herein, is a member that sits beneath a foot. For example, an insole may include the interior bottom of a shoe, a foot-bed, or a removable insert that may be positioned in a shoe or in a sock. Additionally, an “insole” may include a member that is integrated into a sock so that when the sock is worn, the insole is sits beneath the foot. The integration of the accelerometer and pressure sensor into conventional footwear requires minimal extra effort from the user to wear these devices, thus reducing the burden and conspicuousness associated with activity monitoring and facilitating everyday use.

The wearable monitoring system may be based on a combination of multiple sensor modalities, including acceleration and pressure readings from the accelerometer and pressure sensor. The combination of these two modalities may identify many metabolically significant postures and activities. For example, standing can be differentiated from sitting by observing a higher amount of pressure at low levels of acceleration, while walking and jogging may each produce unique patterns of pressure and acceleration at every phase of a gait cycle. Accordingly, the combination of pressure and acceleration data allows for differentiation between major classes of metabolically significant activities, including sitting, standing, walking, jogging, cycling, ascending stairs, descending stairs, household chores, and so on, in which an average person spends the majority of time.

The accelerometer and the pressure sensor may be communicatively connected to a portable or stationary processing device configured to receive and process the data from these devices. For example, the processing device may be configured to automatically determine the user's posture or activity based on the received data, compute energy expenditure based on the type and intensity of the activity, compute performance metrics for different exercise activities (e.g., number of steps, distance for walking or jogging), compute body weight estimates, and/or provide user feedback to maintain a higher metabolic rate. The processing device may be any electronic device having data processing capabilities, and may desirably be a portable device, including a smartphone, a personal digital assistant (PDA), an MP3 player, a laptop computer, table computer or other similar device.

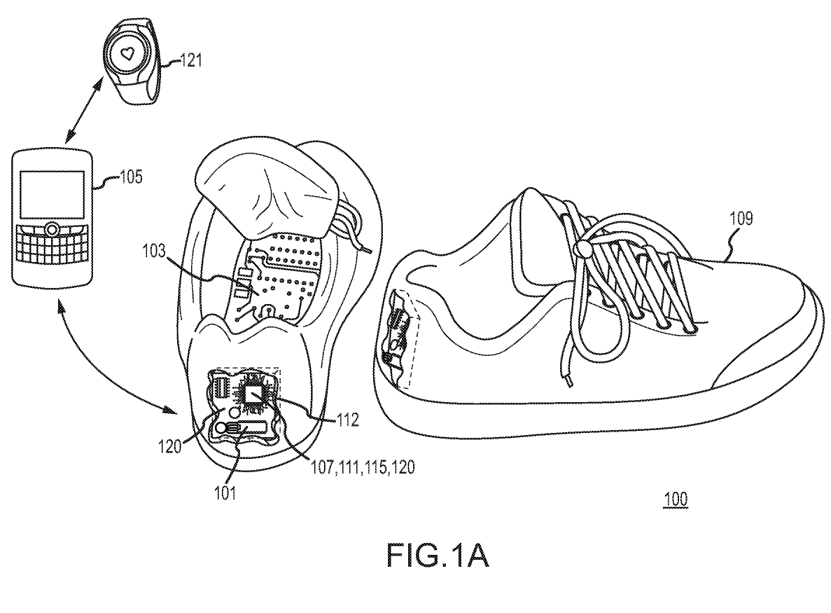

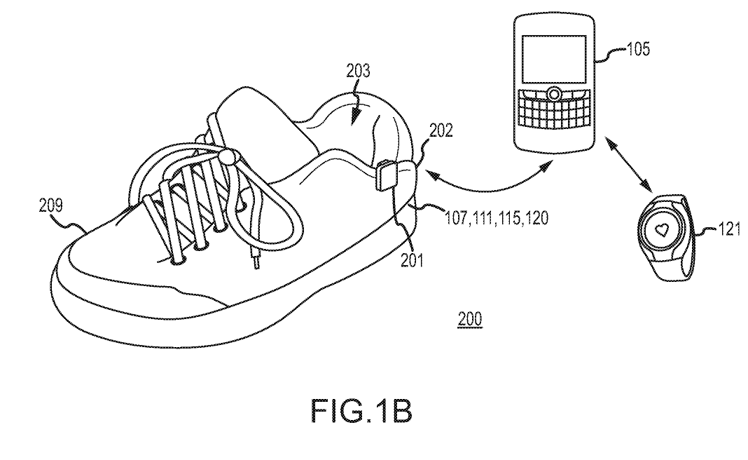

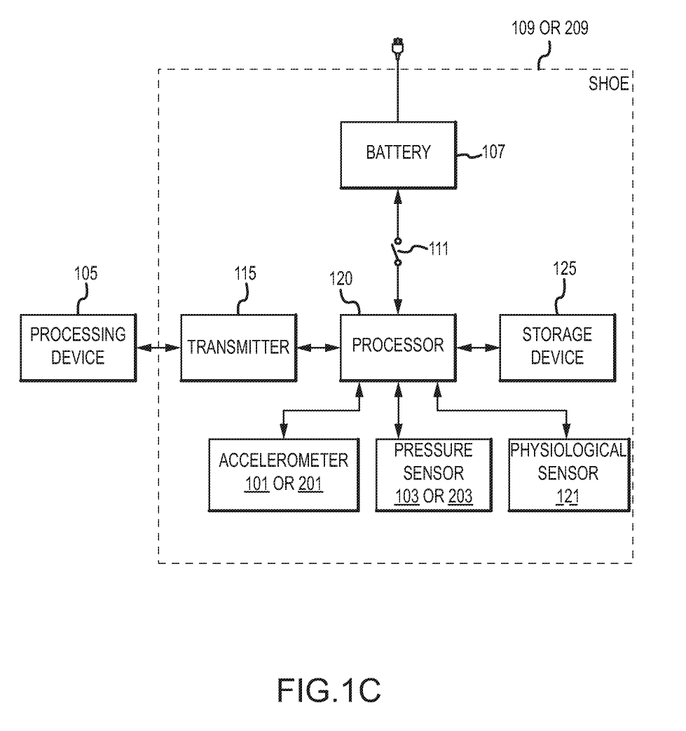

FIGS. 1A and 1B illustrate two embodiments of footwear-based monitoring systems 100, 200, as described herein. Generally, each monitoring system 100, 200 may include an accelerometer 101, 201 and a pressure sensor 103, 203 that are communicatively connected to a processing device 105 configured to process pressure and acceleration data received from the pressure sensor 103, 203 and the accelerometer 101, 201. As will be further described below, the pressure sensor 103, 203 may be integrated into the insole 107, 207 of the user's shoe 109, 209.

In one embodiment, shown in FIG. 1A, the accelerometer 101, 201 may be embedded in the user's shoe 109, 209, for example, in the heel or back portion. In another embodiment, as shown in FIG. 1B, the accelerometer 101, 201 may be provided in a clip-on device 202 that is releasably attachable to the user's shoe 109, 209. In other embodiments, the accelerometer 101, 201 may be otherwise worn by the user. The accelerometer 101 and/or the pressure sensor 103, 203 may be connected to or integrated into or one or both of the user's shoes 109, 209. Accordingly, the monitoring system 100, 200 may be configured to collect acceleration and pressure data from one of the user's shoes 109, 209, or both of the user's shoes. It should be noted that in other embodiments, the accelerometer 101 and/or pressure sensor 103, 203 may be connected to or integrated into the user's clothing, such as the user's socks, or may be independently coupled to the user, such as through an arm band or some other attachment mechanism.

The accelerometer 101, 201 may be configured to measure the physical acceleration experienced by the user's feet. In some embodiments, the accelerometer 101, 201 may be a one-dimensional accelerometer, a two-dimensional accelerometer, or a three-dimensional accelerometer. One example of a two-dimensional accelerometer that may be used in conjunction with the disclosed embodiments is a two-dimensional MEMS accelerometer, which is configured to measure sagittal plane accelerations of the user's feet. An example of a three-dimensional accelerometer that may be used in conjunction with the disclosed embodiments is an LIS3L02AS4 MEMS accelerometer, which is configured to measure accelerations of the user's shoes 109, 209 in three or more orthogonal directions. It should be appreciated that other embodiments may use one or more accelerometers 101, 201 mounted to other portions of the user's shoes or body, and that the accelerometer may sense in other desired planes, such as the coronal plane.

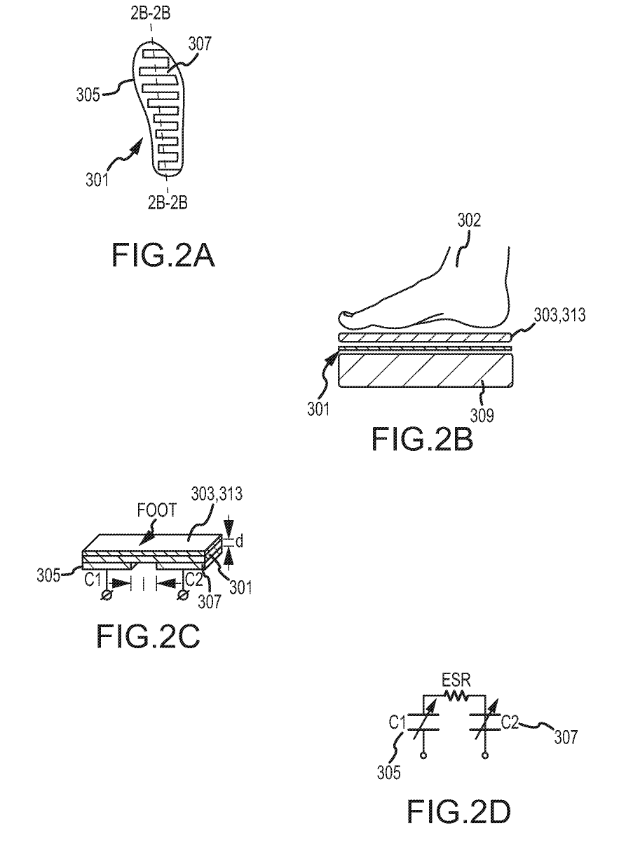

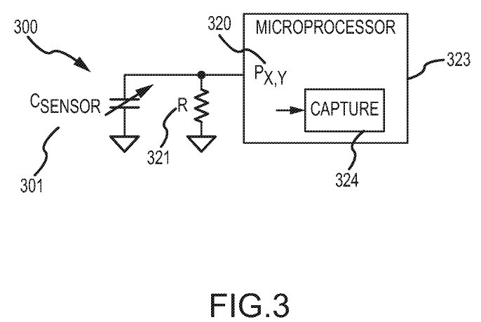

The insole 103, 203 may include a pressure sensor configured to detect changes in the amount of pressure applied to the insole 103, 203 by the sole of the user's feet. The insole 103, 203 may be a flexible insole, and may be configured as a removable insert, incorporated into user's socks, e.g., using a polymer backing or a conductive thread, or attached to the user's shoe 109, 209. As will be further described with respect to FIGS. 2A-2D and FIG. 3, the pressure sensor 101, 201 may be a capacitive sensor or a force-sensitive resistor sensor. The pressure sensor 101, 201 may be configured to detect changes in pressure to identify and differentiate between various parts of the user's gait cycle, including, but not limited to, heel strike, stance phase and toe-off, as well as account for differences in the loading of anterior and posterior areas of the user's foot.

The gait cycle identification and loading profiles obtained from the pressure sensor may be used to classify the type of motion-based activity that the user is performing (e.g., walking vs. running), quantify the amount of body motion in static postures (e.g., shifts in body weight while standing), and distinguish between movement performed along a level surface from movement performed along an inclined (i.e., uphill or downhill) surface, such as a gradually inclined surface, stairs, etc. The gait cycle identification and loading profiles may also be used to detect asymmetries in the gait pattern indicating fatigue or potential development of injury. Additionally, data regarding key temporal and spatial gait parameters, including, but not limited to, cadence, stride length, and stance time, may be extracted from the pressure and/or acceleration data and used to characterize the user's movement-based activities and provide feedback to the user. For example, the feedback may include the number of steps taken by the user, distance walked, cadence, etc.) While the embodiments disclosed herein include capacitive and force-sensitive resistor-based sensing elements, other embodiments may use other pressure sensors to determine the plantar pressure exerted by a user.

Some embodiments of the monitoring system 100, 200 may further include one or more physiological sensors 121 that are also connected to the processing device 105. For example, the physiological sensor 121 may be a bioelectric sensor that is configured to detect electric currents that flow in a user's nerves and muscles, such as the user's heart. In other embodiments, the physiological sensor 121 may be a heart monitor, a piezoelectric pulse monitor, a reflectance optical oximeter configured to detect oxygenation and/or pulse, a respiration sensor, a galvanic skin response sensor, a skin temperature sensor, and so on and so forth. The physiological sensor 121 may be connected to any part of the user's body through either a wired or a wireless connection. For example, the physiological sensor 121 may be positioned directly on the user's skin, over the user's clothing, or in one or both of the user's shoes as an insertable insole, in the user's socks, etc. In one embodiment, the pressure 103, 203 and the physiological sensor 121 can be implemented as a single capacitive sensor. For example, a high impedance capacitive sensor may be used to measure both pressure under the user's feet, as well as bioelectric potential created by the user's heartbeat. These signals may later be separated by signal processing techniques such as frequency filtering, wavelet, or some other transform.

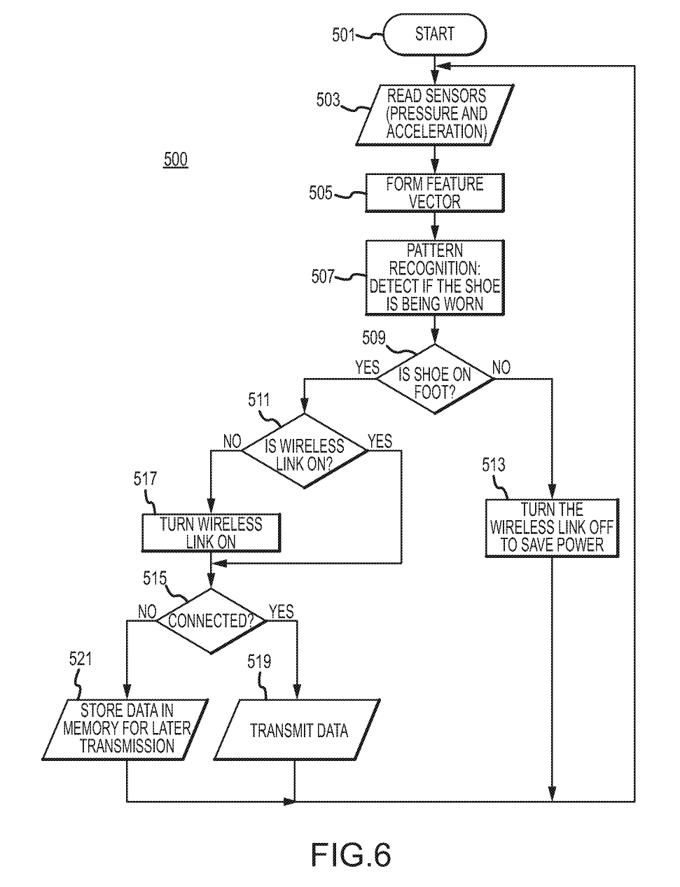

FIG. 1C illustrates a schematic diagram of the monitoring systems 100, 200 shown in FIGS. 1A and 1B. As shown in FIG. 1C, each monitoring system 100, 200 may also include a battery 107, a power switch 111, and a transmitter 115 configured to transmit data to a processing device 105. The monitoring system 100, 200 may further include a processor 120 that is connected to the accelerometer 101, 201, the pressure sensor 103, 203, and any physiological sensors 121. The processor 120 may be configured to sample and process the data collected by the accelerometer 101, 201, pressure sensor 103, 203, and physiological sensors 121 prior to transmission of the sampled data to the processing device 105. Additionally, the processor 120 may be connected to a storage device 125, and may be configured to store sampled data in the storage device 125 for later transmission. Operation of the processor 120 will be discussed in detail with respect to FIG. 6.

In one embodiment, the accelerometer 101, 201, pressure sensor 103, 203, battery 107, power switch 111, transmitter 115, and processor 120 may be coupled to the user's shoes 109, 209. For example, as shown in FIG. 1A, the accelerometer 101, battery 107, power switch 111, and/or transmitter 115 may be installed on the same circuit board 112 and embedded in the heel portion of the user's shoe 109 or integrated into an insole insert that may be positioned under the arch of the user's foot. In another embodiment, as shown in FIG. 1B, the accelerometer 101, battery 107, power switch 111, and/or transmitter 115 may be provided in a clip-on device 202 that can be detached from the user's shoe 209. In yet another embodiment, the accelerometer 101, battery 107, power switch 111, and/or transmitter 115 may be insertable into one of the user's pockets or otherwise attached to user's socks.

Referring to FIG. 1C, the power switch 111 may be configured to activate and deactivate the monitoring system 100, 200 through the processor 120, which may be coupled to the accelerometer 101, 201, the pressure sensor 103, 203, the transmitter 115, and the physiological sensor 121. Power may be supplied by the battery 107, which may be a rechargeable or a non-rechargeable battery, or alternatively, from any AC or DC power source connected to the monitoring system 100, 200, an energy harvester, such as a solar cell, a piezoelectric harvester, and so on and so forth. In an alternative embodiment, the battery 107 may be directly coupled to each or some of the accelerometer 101, 201, the pressure sensor 103, 203, the transmitter 115, and the physiological sensor 121 so as to provide power to these components individually, rather than through the processor 120.

In one embodiment, the monitoring system 100, 200 may also include an activation mechanism configured to allow the user to activate and deactivate the monitoring system 100, 200. The activation mechanism may be a mechanism provided on the user's shoe 109, 209, such as a switch, button, lever, motion sensor, pressure sensor (resistive or capacitive), etc. or may be a device that is remotely connected to the monitoring system 100, 200, such as a remote control, a remote motion sensor, etc.

As discussed above, the pressure sensor 103, 203 may be configured to activate and deactivate the monitoring system 100, 200. For example, the pressure sensor 103, 203 may be configured to activate the monitoring system 100, 200 when the user is wearing the shoes 109, 209, i.e., when pressure is applied to the pressure sensor 103, 203, and deactivate the monitoring system 100, 200 when the user is not wearing the shoes 109, 209, i.e., when no pressure is applied to the pressure sensor 103, 203. In other embodiments, the pressure sensors 103, 203 may further be configured to place the monitoring system 100, 200 into a low-power, or “sleep” state when the sensors 103, 203 determine that the user is not wearing one or both shoes 109, 209. The “sleep” state may serve to prolong the battery life of the monitoring system, and may further serve to expedite the time required for activating the monitoring system 100, 200.

The transmitter 115 may be connected to the processor 120 of the monitoring system 109, 209, and may be configured to transmit sampled pressure and acceleration data collected by the accelerometer 101, 201, the pressure sensor 103, 203, and/or the physiological sensor 121 to a processing device 105 that is configured to process the received data. The data transmission may be through either a wired or a wireless transmission medium. In one embodiment, the transmitter 115 may be a wireless transmitter, and may use a wireless protocol for communicating with the processing device. For example, the transmitter 115 may use an a Bluetooth wireless protocol, an ANT protocol, or a ZigBee protocol. In one embodiment, the wireless protocol may be a low-power consumption protocol that preserves the battery life of the battery 107.

The processing device 105 may be a dedicated electronic device or a ubiquitous electronic device that is configured to perform other functions. Some examples of electronic devices that may be used in conjunction with the disclosed embodiments include, but are not limited to, a personal computer, such as a laptop, tablet PC or a handheld PC, a PDA, a mobile telephone, a media player, such as an MP3 player, or a television receiver. As will be further discussed below, the processing device 105 may run monitoring software configured to process the collected data and provide feedback to the user regarding the collected data. For example, the monitoring software may use the collected acceleration and pressure data to calculate the weight and energy expended by the user and provide this information to the user as feedback.

As discussed above, FIG. 1A illustrates an embodiment in which the accelerometer 101, battery 107, power switch 111, and/or transmitter 115 are installed on a circuit board 112 that is embedded in the heel portion of the user's shoe 109. As shown in FIG. 1A, the circuit board 112 may connected to the pressure sensors 101 at the tail end portion of the insole. For example, the tail end portion of the insole may be fed through a narrow cut in the shoe and connected to the bottom end of the circuit board. In other embodiments, the circuit board 112 may be embedded into another portion of the shoe 109, or may be glued, sewn, or otherwise attached to the interior or the exterior of the shoe 109. Additionally, the circuit board need not be physically coupled to the pressure sensor, but may be wirelessly or otherwise communicatively connected to the pressure sensor.

As discussed above, FIG. 1B illustrates an embodiment in which the accelerometer 201, battery 107, power switch 111, and/or transmitter 115 are provided in a device 202 that is releasably attached to the shoe 209. The device 202 may include a clip or some other releasable attachment mechanism that allows a user to conveniently attach and remove the device 202 from the shoe 209 or sock. For example, other embodiments of the device may include one or more bores that allow the user to tie the device to his or her shoelaces. In another embodiment, the attachment mechanism may be a band that is adjustable in size so as to allow the user to attach the band around the user's ankle, leg, arm, or some other body part. Other attachment mechanisms are also possible, as are well known in the art.

The accelerometer, battery, power switch, and/or transmitter may be more or less distributed in other embodiments. For example, the accelerometer and the transmitter may be integrated into the shoe, while the battery and the power switch maybe provided on a separate device.

FIGS. 2A-2D illustrate an embodiment of a capacitive sensor 301 integrated into an insole 303. As shown in FIGS. 2A and 2B, the capacitive sensor 301 may include a first conductor layer including one or more conductive plates 305, 307. The plates 305, 307 may be embedded in the insole 303 between the top insole layer 313 and the sole of the shoe 209. Alternatively the plates 305, 307 may be configured as a conductive thread or a polymer that is incorporated into an removable insole 303 or a sock. As shown in FIG. 2A, the conductive plates 305, 307 may span the entire surface area of the insole 303 or, in other embodiments, may span only a portion of the insole 303. A larger surface area may correlate to a more accurate capacitance reading and an increased sensitivity of the capacitive sensor 301. A sensor covering only a portion of the insole may be used to measure pressure under specific areas of interest such as the user's heel, metatarsal heads, big toe, etc. The sole of the user's foot 302 may function as a second conductive layer so that a potential difference is created between the plates 305, 307 and the user's foot 302 when the capacitive sensor 301 is activated. Accordingly, the top insole layer 301, which is positioned between the plates 305, 307 and the user's foot 302 may function as a dielectric layer, and may be formed from rubber foam, or some other non-conductive material. The top insole layer 301 may relatively thin and flexible so as to increase the sensitivity of the plates 305, 307 with respect to the user's foot 302. As an example, in one embodiment, the insole may be between 1-5 mm thick.

As shown in FIGS. 2A and 2C, the first conductive layer may include two conductive plates 305, 307 that are connected in series. In one embodiment, the plates 305, 307 may each have a comb-like structure, and may be interleaved so as to minimize the Equivalent Series Resistance (“ESR”) in the tissue of the user's foot 302 and thus provide for a more accurate measurement. Additionally, the plates 305, 307 may be separated by a predetermined distance to place more tissue into equivalent electrical contact and further reduce the ESR in the user's foot. As an example, the grid step size, i.e., the width of the portion of each plate forming a tooth of the comb-like structure, may be between 0.25 cm to 2 cm.

Referring to FIG. 2C, the conductive plates 305, 307 may serve as the first conductive layer of a capacitive sensor 301 and the sole of the user's foot 302 may serve as the second conductive layer of the capacitive sensor 301 so that pressure exerted onto the top insole layer 313 by the user's foot 302 changes the distance d between the plates 305, 307 and the sole of the user's foot 302. Accordingly, when the user's foot 302 applies more pressure to the top insole layer 313, the gap d between the sole of the user's foot 302 and the conductive plates 305, 307 becomes smaller, and the capacitance between the user's foot 302 and the plates 305, 307 increases. Similarly, when the user's foot 302 applies less pressure to the top insole layer 313, the gap d between the sole of the user's foot 302 and the conductive plates 305, 307 becomes larger, and the capacitance between the user's foot and the plates 305, 307 decreases. In one embodiment, the capacitance between the user's foot 302 and the plates 305, 307 may be expressed as C=ε_{y}ε₀γ_{d}, where ε_{y} is relative static permittivity (dielectric constant) of the dielectric layer, e.g., top insole layer 307,

$${ɛ}_{o} = 8.854 E - 12 \frac{F}{m}$$

is the permittivity of free space, A is the area of overlap between plates in m², d is the distance between plates in m.

The capacitive sensor may provide data similar to that provided by a force sensitive resistor sensor. For example, the changes in capacitance of the sensors may be proportional to the pressure applied by user in static postures and dynamic activities. Thus, the changes in capacitance can be used to identify various parts of gait cycle, amount of body weight shifting (fidgeting) in static postures, and/or be used for weight measurement. As an example, the capacitive sensor may be configured to sense a significant change, i.e., increase, in capacitance when a user's heel strikes the ground, a decrease in capacitance during the middle of a stance, and an increase in capacitance during the end of stance.

FIG. 3 illustrates an embodiment of a circuit (resistor-capacitor circuit 300) that is configured to perform capacitive pressure sensing using the capacitive sensor 301 shown in FIGS. 2A-2D. As shown in FIG. 3, the circuit 300 may include the capacitive sensor 301, a processing device 323, and a resistor 321. In one embodiment, the capacitive sensor 301, processing device 323, and resistor 321 may be integrated into the user's shoe, such as in the insole, the user's socks, etc.

As shown in FIG. 3, data obtained from the capacitive sensor 301 may be processed by a processing device 323, such as an MSP430 microcontroller or other processing device, for example, an AVR or PIC microcontroller. The processing device 323 may be configured to measure the discharge time of the resistor-capacitor circuit 300, which, as discussed above, includes a capacitive pressure sensor 301 and a resistor 321. In one embodiment, a general-purpose pin 320 in an output mode may charge the capacitive sensor 301 to a known voltage. A timer including a capture register 324 may be set and the pin 320 may be switched to an input mode. The capacitive sensor 301 may then discharge though the resistor 321, which may have a known resistance R. When the voltage of the capacitive sensor 301 crosses the low threshold voltage of the pin 320, an internal interrupt may be generated that stops the timer. The captured number of timer clicks, i.e., the discharge time, is proportional to the capacitance of the capacitive sensor C, which may be between 64.5-387 pF. The discharge time of the RC circuit to near ground may be approximately T_{DISCHARGE}≈5_{T}≈5RC.

As an example, if the resistance R is approximately 1 Mohm, the discharge time may vary between 322 uS to 1.9 mS for sampling frequencies greater than 500 Hz. When in an input configuration, the MSP430 microcontroller may have a ±50 nA leakage port current that is negligible as compared to the discharge current through the resistor R (3 uA at 3V), and thus does not impact the accuracy of the readings. The ESR of the capacitive sensor may also be taken into consideration if necessary, i.e., if the ESR is high enough to influence the discharge time of the capacitive sensor. In one embodiment, a 16-bit timer may be clocked using a 16 MHz crystal, which may result in 5000 to 30400 counts per measurement. The resulting discretization of the capacitance is fine enough to capture even minute variations in pressure and/or weight, as applied to the capacitive sensor.

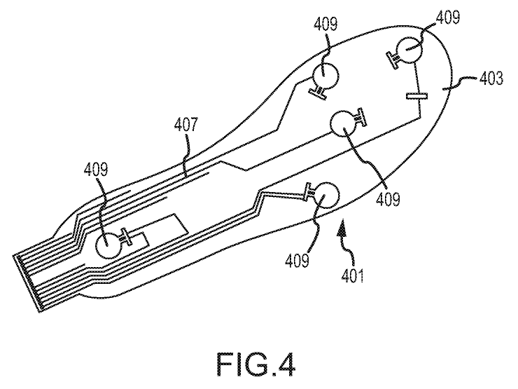

FIG. 4 illustrates an alternative embodiment of an insole 403 that may use force sensitive resistor pressure sensors 401. As shown in FIG. 4, the insole 403 may include a flexible printed circuit board 407 that support multiple force sensitive resistors 409. The force sensitive resistors 409 may consist of a conductive polymer that changes resistance in a predictable manner in response to the application of force to its surface. More particularly, the force sensitive resistors 409 may include both electrically conducting and non-conducting particles suspended in matrix. Applying a force to the surface of a resistors 409 causes particles to touch conducting electrodes, changing the resistance of the resistor 409.

In one embodiment, the insert may include five total resistors 409 positioned under various points of contact with the user's foot, including the heel, heads of metatarsal bones and the big toe. While five sensors are illustrated, it should be appreciated that any number of force sensitive resistors 409 may be distributed in any configuration throughout the insole, so long as the pressure sensor 401 provides sufficient information to recognize and characterize postures, activities and/or measure weight. Additionally, the size of the resistors 409 may vary in other embodiments. For example, a single force sensitive resistor may be large enough to cover both a metatarsal bone and the toe.

The positioning of the force-sensitive resistors may allow for differentiation of various parts of the user's gait cycle. For example, a pressure sensor under the heel may serve to detect the initial contact of the foot with the ground (i.e. heel strike). Additionally, various amounts of pressure on the heel and metatarsal sensors, in combination with acceleration readings, may suggest a particular stance phase of the user's gait cycle, and so on and so forth.

The number of pressure sensors 401 may vary from embodiment to embodiment. For example, one embodiment may use a single pressure sensor covering the entire area under the foot or a portion of the area under the foot, while another embodiment may use 34 pressure sensors that are positioned at various locations under the user's foot. One particular sensor layout includes 3 pressure sensors: a pressure sensor that is positioned under the user's heel, a pressure sensor that is positioned under the user's metatarsal heads, and a pressure sensor that is positioned under the user's big toe. Another sensor layout includes a multitude of sensors, for example between 25 to 100 sensors, that are evenly distributed under the foot.

The layout of the sensors is not dependent on the sensor type, i.e., whether the sensor is a resistive or a capacitive pressure sensor, but may instead be selected based on the desired functionality and accuracy of the overall pressure sensor. For example, different layouts and/or numbers of sensors may have varying impacts on functionality, accuracy, and implementation costs.

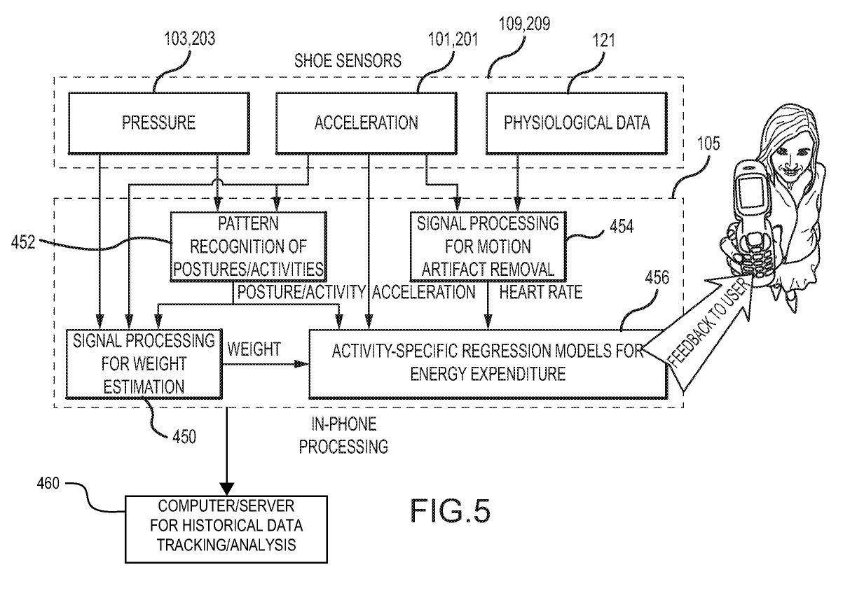

FIG. 5 is a schematic diagram illustrating information flow within an embodiment of the monitoring system. As shown in FIG. 5, data signals from the pressure sensor, accelerometer, and physiological sensors may be transmitted by a wired or a wireless connection to a processing device. The pressure and physiological potential sensors can be implemented as a single sensor or as separate sensors, but are shown in FIG. 5 as separate sensors for clarity.

The data signals from the pressure sensors, accelerometer, and physiological sensors may transmitted to different processing modules, which may apply signal processing, pattern recognition, and classification algorithms to the received data to measure weight, recognize postures and activities, estimate energy expenditure, and provide feedback to a user. As shown in FIG. 5, the electronic device may run monitoring software including various software modules that are configured to receive data from some or all of the pressure sensors, accelerometer, and physiological sensors. For example, the processing device may include a posture and/or activity pattern recognition module 452. The pattern recognition module 452 may receive signals from the pressure sensor, accelerometer, and/or physiological sensor to recognize postures, for example, whether the user is sitting, standing, or in another posture, and movement-based activities, such as whether a user is walking, jogging, cycling, and so on.

Additionally, the processing device may include a weight estimation module 450 that is configured to receive information about the user's posture and/or activity from the activity pattern recognition module 452, as well as acceleration and pressure data from the accelerometer and pressure sensor, to compute an estimate of the user's weight. In one embodiment, the user's weight can be measured as proportional to pressure when the device detects a standing posture, and acceleration data indicates that the user is in a quiet standing position, for example, if the acceleration data indicates that the acceleration of the user is below a fidgeting threshold.

The signal processing module 454 may be configured to receive physiological data and extract various metrics of interest, such as the user's heart rate. Additionally, the signal processing module 454 may be configured to receive and process acceleration data from the accelerometer to remove to remove signal artifacts that may be created by user's movements.

The processing device 105 may further include an energy expenditure estimation module 456 that is configured to receive acceleration data from the accelerometer 109, 209, as well as processed data from the signal processing module 454, and apply one or more predictor values to calculate the user's energy expenditure. The predictor values may include, but are not limited to, the user's weight, height, current posture or activity, features derived from pressure and/or acceleration data, the user's heart rate, and so on and so forth. In one embodiment, the energy expenditure estimation module may also be configured to monitor the time that a user is performing a particular activity or holding a particular posture, or if the user's energy expenditure level is below a set target for a predetermined period of time. Where such an event is detected, the device 105 may be configured to proactively alert the user and/or suggest corrective actions to boost the user's energy expenditure.

In another embodiment, the processing device may be configured to allow the user to retrieve both historical and current data on demand. It should be noted that other embodiments may include more or fewer software processing modules. For example, the weight estimation module 450 may be replaced by an application that is configured to allow users to enter their weight through a Graphical User Interface. As another example, the physiological sensor 121 may be absent from the system and heart rate may not used in the energy expenditure calculation. Other combinations of sensors and/or processing modules are also possible.

Additionally, the processing device may be connected to other processing devices running the monitoring software, for example, through a network. A “network,” as used herein, is a group of communication devices connected to one another and capable of passing data therebetween. As such, a network may be the Internet, a computer network, the public-switched telephone network, a wide-area network, a local area network, a cellular network, a global Telex network, a cable network or any other wired or wireless network.

In this embodiment, the monitoring software may be stored on a separate server 460 connected to the network, rather than on the processing devices, e.g., 105, themselves, and the processing devices may be configured to access the monitoring software through the server 460. In another embodiment, the server 460 may be configured to host a community website that users can access through the processing devices to compare their posture and movement-based activity data to statistics from other individual users and to the user population in general. The website may also be configured to host competition-based weight maintenance/loss/gain programs based on data collected from each user's monitoring system. These and other functions may be accessed and managed by a user via a graphical user interface (“GUI”). For example, the GUI of the monitoring software may permit users to add contacts, create web groups, schedule meetings, and so on and so forth.

FIG. 6 illustrates a flow diagram of a method 500 for monitoring energy expenditure, body weight, posture allocation, and physical activity classification using pressure and acceleration data. In the operation of block 501, the method begins. In some embodiments, the method may be executed by a processor that is coupled to the user's shoe. In other embodiments, the processor may reside on a separate computing device that receives data transmitted (e.g., wirelessly from the sensors in the shoe. The processor may be communicatively coupled to one or more pressure sensors and one or more accelerometers. In other embodiments, the processor may also be communicatively coupled to one or more physiological sensors, or other personal monitoring devices. In the operation of block 503, the processor may be configured to read the data collected from the pressure sensor and the accelerometer.

In the operation of block 505, the processor may be configured to sample the data and form a feature vector from the collected data. In one embodiment, the processor may obtain multiple readings, compute derived features, and combine them into a feature vector that includes several lagged measurements of acceleration and/or pressure. As an example, the pressure and acceleration data may be sampled at 25 Hz by a 12-bit analog-to-digital converter. In one embodiment, data acquisition may be based on a Wireless Intelligent Sensor and Actuator Network (“WISAN”) that is configured for time-synchronous data acquisition. Accordingly, the WISAN may allow for data sampling at substantially the same time (with a difference of no more than 10 microseconds) from two shoes, as worn by the user. In another embodiment, data acquisition may be based on a circuit that combines a microcontroller equipped with an analog-to-digital converter (and/or an SPI or 12C interface for reading sensor signals) and a Bluetooth or a Bluetooth Low Energy module for wireless transmission of the data. In yet another embodiment, the microcontroller may be configured to transmit the sensor data through a standardized (for example, Zigbee, ANT, and so on) or custom (for example, based on an nRF24LE01 chip or a similar chip) wireless interface.

In the operation of block 507, pattern recognition is performed on the feature vectors to determine if the user is wearing the shoe. An example of pattern recognition algorithm may be a simple threshold classifier that is configured to determine that the user is wearing a shoe when the pressure reading from the pressure sensor exceeds a predefined threshold. Another example of a pattern recognition algorithm may determine that the user is wearing a shoe when the collected acceleration data indicates that the motion of the shoe exceeds a predefined threshold. In other embodiments, both acceleration and pressure data may be used to determine whether the user is wearing the shoe. For example, other algorithms may include artificial neural networks, support vector machines, and other classification algorithms.

If, in operation 509, the processor determines that the user is wearing the shoe, then in the operation of block 511, the processor determines whether the wireless transmitter needs to be turned on. If in operation of 511 the processor determines that the wireless link is off, then, in the operation of block 517, the wireless link is turned on.

If, in the operation of block 509, the processor determines that the user is not wearing the shoe, then, in the operation of block 513, the processor may turn off the wireless transmitter to save power. Additionally, the processor may further be configured to turn off any sensors and/or other electronic components of the monitoring system.

If, in the operation of block 511, the processor determines that the wireless transmitter is turned on, then, in the operation of block 515, the processor may determine whether the transmitter is connected to the processing device. This may include determining whether the receiver in the processing device is turned on and that the receiver is enabled to receive data from the transmitter. If, in the operation of block 511, the processor determines that the wireless transmitter is turned off, then, in the operation of block 517, the processor may turn on the wireless link, and, in operation 515, determine whether the transmitter is connected to the processing device.

If, in the operation of block 515, the processor determines that the transmitter is connected to the processing device, then, in the operation of block 519, the transmitter may transmit the pressure and acceleration data to the receiving processing device. In one embodiment, the pressure and acceleration data may be sampled at a higher rate than when the monitoring system is in an inactive mode. If, however, in the operation of block 515, the processor determines that the transmitter is not connected to the processor device, then, in the operation of block 521, the processor may store the data in a storage device for later transmission. For example, the processor may store the data until it determines that the transmitter is connected to the processor device, at which point it may retrieve the data from the storage device and transmit the data to the receiving processing device.

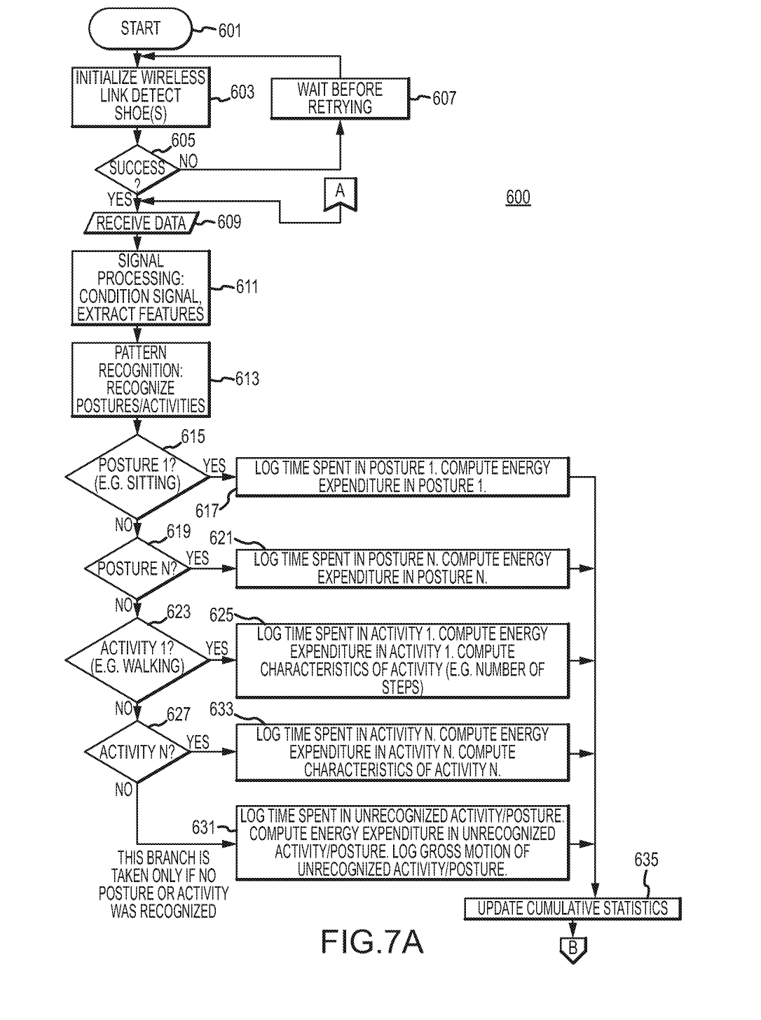

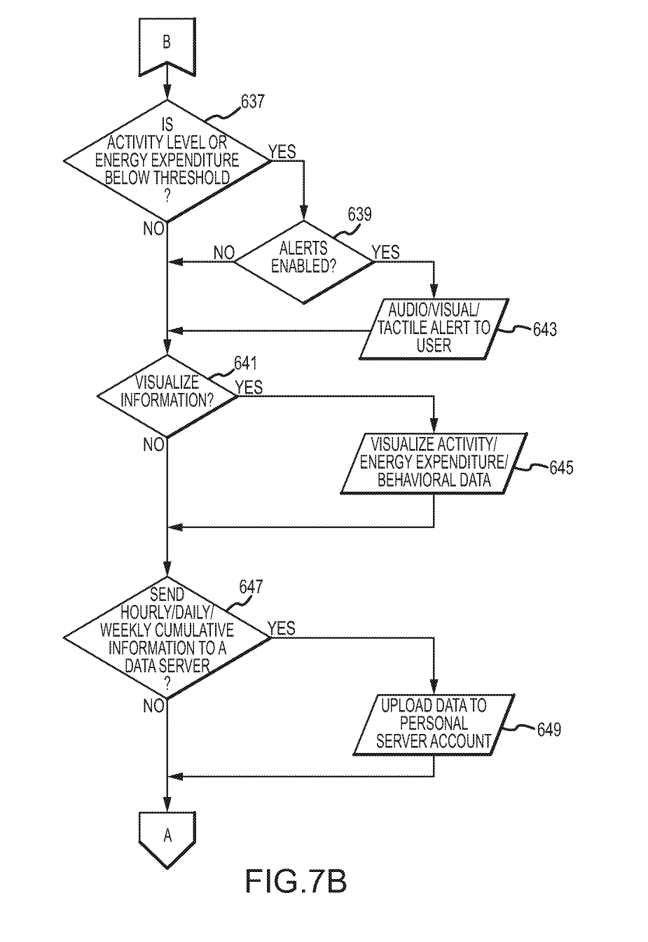

FIGS. 7A-7B illustrate a method 600 for monitoring energy expenditure, body weight, posture allocation, and physical activity classification using pressure and acceleration data, as executed on a processing device that is communicatively coupled to the accelerometer, pressure sensor and/or physiological sensor. As discussed above, the processing device may be a portable electronic device, such as a PDA, a laptop, a cellular phone, a dedicated device specifically designed to provide feedback to the user, and so on. In the operation of block 601, the method may begin. In the operation of block 603, the processing device may initialize the wireless link to the pressure sensors and accelerometer (as well as any physiological or other sensors) or data collected by these sensors and saved for transmission to the processing device. In the operation of block 605, the processing device may determine whether the wireless link was successfully established. If, in the operation of block 605, the processing device was not successfully connected to the sensors, then, in the operation of block 607, the processing device may wait for a period of time before trying to reinitialize the wireless link.

If, in the operation of block 605, the processing device determines that the wireless link was successfully established, then, in the operation of block 609, the processing device may receive data transmitted from the accelerometer and the pressure sensor. In the operation of block 611, the processing device may use signal processing techniques to condition the received data signals, as well as extract relevant features. Examples of signal processing techniques that may be used include normalization of data to a specified range of values, formation of lagged vectors representing a time slice of the signals, computation of derived metrics such as room-mean-square, entropy, spectral coefficients, and so on.

In the operation of block 613, the processed signals may be further processed to use pattern recognition to recognize various postures and/or movement-based activities of the user. For example, the processing device may be configured to determine whether the user is sitting, standing, walking, etc. by applying pattern recognition algorithms to the received data signals. Pattern recognition algorithms that may be used include artificial neural networks, for example, multi-layer perceptron, or other classification algorithms, such as support vector machines, Bayesian classifiers, etc. A feature vector may be presented to the pattern recognition algorithm, which may assign it to one of the classes ('sitting', ‘standing’, etc) based on previously learned examples. For example, low values of acceleration combined with a pressure reading that is less than the user's body weight indicate that the user is sitting, while low acceleration values combined with a pressure reading that is substantially equal to the user's body weight indicate that the user is standing. Walking may be characterized by horizontal and vertical acceleration patterns that exhibit low cycle-to-cycle variability, combined with pressure changes that alternate between high and low (stance/swing) and travel from heel to toe.

The posture or activity represented by the feature vector may not belong to the list classes known to the pattern recognition algorithm. For example, the pressure and/or acceleration readings may not match a posture or activity that is readily classifiable by the processing device. In such cases, the classification may be performed with a hard assignment in which an unknown posture or activity is assigned to the closest classifiable posture or activity, or, in other embodiments, the classification may be performed with a rejection in which the unknown posture or activity is classified as an unclassifiable posture or activity.

In the operation of block 615, the processing device may determine whether the posture is the first posture within a list of predetermined postures. For example, the processing device may determine whether the user is sitting or standing. If, in the operation of block 615, the processing device determines that the posture is the first posture, then, in the operation of block 617, the processing device may log the time spent in the first posture and compute the estimated energy expended by the user in the first posture. The computation of energy expenditure may be performed using a linear or non-linear regression utilizing one or more predictors, such as weight (either measured or entered by the user), height, age, body-mass-index, heart rate, and metrics derived from pressure and acceleration signals such as mean, root-mean-square, standard deviation, coefficient of variation, entropy, number of zero crossings, including metrics after logarithmic transform and metrics for combinations of signals (for example, as a sum or product). To improve the accuracy of the energy expenditure estimation, a dedicated regression model may be built for a specific posture class known to the classifier (e.g. ‘sitting’). The predictors and the regression equations may vary from posture to posture.

If, in the operation of block 615, the processing device determines that the posture is not the first posture in the predetermined list of postures, then, in the operation of block 619, the processing device may determine whether the posture performed by the user is another posture in the predetermined list of postures. If, in the operation of block 619, the processing device determines that the posture is the another posture in the predetermined list of postures, then, in the operation of block 621, the processing device may log the time that the user is performing the posture and calculate the energy expended by the user while in the posture.

If, however, in the operation of block 619, the processing device determines that the posture is not in the predetermined list, then, in the operation of block 623, the processing device may be configured to determine whether the user is performing a first movement-based activity within a list of predetermined movement-based activities. As discussed above, some activities may include, for example, walking, cycling, running, climbing stairs, and so on. If, in the operation of block 623, the processing device determines that the user is not performing the first activity, then, in the operation of block 627, the processing device may be configured to determine whether the user is performing another activity in the list. If, in the operation of block 623, the processing device determines that the user is performing the first activity, then, in the operation of block 625, the processing device may be configured to log the time that the user spends performing the activity and compute the energy expended by the user while performing the activity.

In one embodiment, energy expenditure may be computed by a linear or non-linear regression model that uses one or more predictors such as weight (either measured or entered by the user), height, age, body-mass-index, heart rate, and/or metrics derived from pressure and acceleration signals. To improve accuracy of energy expenditure estimation, a dedicated regression model may be built for a specific activity class known to the classifier (e.g. ‘walking’). The predictors and the regression equation may vary from activity to activity. For example, each activity may have an associated energy expenditure model. Additionally, each activity may include an associated “intensity” to more accurately estimate the energy expenditure of the user. For example, if the energy expenditure associated with walking at 2.5 mph is 4 kcal/min, then this value is used when the classifier identifies the activity as walking at an intensity of 2.5 mph. Additionally, the processing device may be configured to compute one or more characteristics of the activity, for example, the number of steps taken by the user, and use this information to compute the energy expended by the user in performing the activity.

If, in the operation of block 627, the processing device determines that the user is not performing a movement-based activity in the list, then, in the operation of block 631, the processing device will categorize the activity or posture as an unclassifiable or unrecognized activity or posture, and compute the energy expended by the user in performing the unrecognized activity or holding the unrecognized posture. For example, the acceleration and/or pressure data may be used to model the energy expenditure of the user, rather than classifying the activity or posture being performed by the user. In other embodiments, the processing device may assign a generic energy expenditure value that can be used to compute the energy expended by the user in performing the unrecognized activity or posture. These energy expenditure calculations may be performed by a regression model, as previously discussed above with respect to known activities and postures. Alternatively, the processing device may use the acceleration and/or pressure data to classify the activity or posture of the user into one of the known activities or postures. However, the regression model for the unclassifiable activity or posture may encompass a range of postures and activities and may therefore be less accurate than activity- or posture-specific models.

If, in the operation of block 627, the processing device determines that the user is performing another activity on the list, then, in the operation of block 633, the processing device may be configured to log the time that the user spends performing the recognized activity and compute the energy expended by the user while performing the activity. For example, the processing device may be configured to compute one or more characteristics of the sensor signals (such as metrics for the regression models described above) or characteristics of the activity being performed (for example, cadence and/or number of steps). Other characteristics may include the associated posture, intensity of the acceleration signal (magnitude and frequency) and/or the magnitude of the pressure readings.

In the operation of block 635, the processing device may add the calculated energy expenditures for each of the recognized and unrecognized postures and/or activities to the user's prior calculated energy expenditures to obtain a cumulative energy expenditure statistic for a predefined period of time. This may be done periodically, for example, every minute or each time that the system determines that the user is performing a new activity or holding a new posture. Additionally, the time period may vary from embodiment to embodiment, or may be user selected. For example, the cumulative period may be a day, an hour, a week, and so on.

In the operation of block 637, the processing device may determine whether the activity level or energy expenditure for the user is below a predefined threshold. The threshold may be calculated by the processing device, for example, based on the user's weight and a target weight, target energy expenditure for a person of certain anthropometric characteristics, input by the user, or obtained by some other means. If, in the operation of block 637, the processing device determines that the energy expenditure is below the threshold or the user has been assuming a static posture for too long, then, in the operation of block 639, the processing device may determine whether the alerts for notifying the user that his or her energy expenditure is below the threshold have been enabled. If, in the operation of block 637, the processing device determines that the energy expenditure of the user meets or exceeds the threshold, then, in the operation of block 641, the processing device may determine whether or not a visualization of the user's energy expenditure data should be generated and provided to the user. For example, the processing device may determine whether or not the user has prompted the processing device for a visualization of his or her energy expenditure data.

If, in the operation of block 639, the processing device determines that the alerts for notifying the user that his or her energy expenditure is below the threshold have been enabled, then, in the operation of block 643, the processing device may provide an audio, tactile, and/or visual alert to the user. For example, the processing device may generate a pop-up icon or sound that notifies the user as to his or her failure to meet the threshold. In addition, the processing device may offer a suggested corrective action. For example, the processing device may generate an alert advising the user to “take at least 100 steps more a day,” and so on.

If, in the operation of block 641, the processing device determines that a visualization of the user's energy expenditure should be generated, then, in operation 645, the processing device may generate a visual depiction of the user's cumulative energy expenditure, activity, and/or behavioral data. This may include any graphs and/or charts summarizing this information. If, in the operation of block 641, the processing device determines that a visualization of the user's energy expenditure should not be generated, then, in operation 647, the processing device may determine whether it should periodically send cumulative energy expenditure, activity, and/or behavioral data to a data storage device. This feature may be enabled by a user, for example, by manipulating settings through the graphical user interface of processing software running on the processing device. The data storage device may be a remote data server, or in other embodiments, may be a memory device within the processing device. If, in operation 647, the processing device determines that it should periodically send cumulative energy expenditure, activity, and/or behavioral data to a data storage device, then, in operation 649, the processing device may be configured to upload the data to the user's personal server account. If, in operation 647, the processing device determines that it should not periodically send cumulative energy expenditure, activity, and/or behavioral data to a data storage device, then, the method returns to operation 609.

### I. FIRST EXPERIMENT (Using a Force Sensitive Resistor Pressure Sensor)

A. Data Collection

1. Shoe-Based Wearable Sensor

The plantar pressure and heel acceleration data were collected by a wearable sensor system embedded into subjects' shoes. Each shoe incorporated five force-sensitive resistors (Interlink Inc.) integrated with a flexible insole and positioned under the critical points of contact: heel, heads of metatarsal bones and the big toe. Such positioning allowed for differentiation of the most critical parts of the gait cycle such as heel strike, stance phase and toe-off as well as accounting for differences in loading of anterior and posterior areas of the foot in ascending/descending stairs and cycling. In an alternative configuration, not used in these studies, a clip-on sensor device may be attached to a shoe. The motion information was provided by a 3-dimensional accelerometer (LIS3L02AS4) positioned on the back of the shoe. The goal of accelerometer was to detect orientation of the shoe with respect to gravity, to characterize the motion trajectory and to help characterize fidgeting in static postures as well as intensity of physical activity. The battery, power switch and wireless board were installed on a rigid circuit board glued to the back of the shoe. The tail of the flexible insole was fed through a narrow cut in the shoe and connected to the same circuit board. The sensor system was very lightweight and created no observable interference with motion patterns.

Pressure and acceleration data were sampled at 25 Hz by a 12-bit analog-to-digital converter and sent over a wireless link to the base computer. The wireless system used for data acquisition was based on Wireless Intelligent Sensor and Actuator Network (WISAN) developed specifically for time-synchronous data acquisition. Application of WISAN allowed for data sampling at exactly the same time (with a difference of no more than 10 microseconds) from both shoes. The sensor data were streamed to a portable computer with a Labview front end and stored on the hard drive for further processing.

2. Data Collection Protocol

Data collection was performed on a group of 16 human subjects, 8 males and 8 females. Institutional Review Board approval and each subject provided informed consent. The subjects were chosen to reflect a diverse adult population. Subject characteristics were: mean age of 25±6.5 years (range 18-44); mean weight was 76.9±20.6 kg (range 48.6-119.8), mean Body Mass Index (BMI) was 26.7±6.5 kg/m² (range 18.1-39.4). The shoe sizes (US) ranged from 9.5 -11 for men and from 7-9 in women. Based on self-report, volunteers were weight stable (<2 kg weight fluctuation) over the previous 6 months. Individuals that were healthy, non-smokers who were sedentary to moderately active (<2-3 bouts of exercise/wk or participation in any sporting activities <3 hr/wk) were invited to participate in the study. Pregnant women and those who had impairments that prevented physical activity were excluded

Data collection for each subject was performed during a single 2.5-3 hour visit. The subjects wore the sensor-equipped shoes for the duration of the visit. The subjects also wore a portable metabolic system (Viasys Oxycon Mobile) to measure energy expenditure. The data collection protocol is shown in Table 1. A total of 20 hours 37 minutes of data were recorded for 6 major posture/activity classes: sitting motionless or with fidgeting (3 hr 9 min), standing motionless or with fidgeting (3 hr 5 min), walking/jogging at various speeds and grades (10 hr 33 min), ascending stairs (36 min), descending stairs (32 min) and cycling at 50 and 75 rpm (2 hr 34 min). Recognizing these 6 classes from the shoe sensor data was one of the major goals of this study. Subjects were not restricted in the way they assumed postures and or performed activities. Standing did not require any specialized equipment; a chair with a rigid back was used for sitting; walking/jogging was performed on Biodex Gait Trainer 1 treadmill; subjects used stairs between the ground and second floor for ascending and descending; cycling utilized Ergomedic 828E bicycle exerciser:

TABLE 1
Protocol of the data collection

| Item | Description | Duration |
|---|---|---|
| 1 | Initial Interview, signing of the informed | 20 min |
| | consent, fitting of the sensors | |
| 2 | Sit/Stand motionless | 12 min (6 min each) |
| 3 | Walking/Jogging: 6 minute trials, 5 minute | 44 min |
| | rest between trials. Four level trials: 1.5, | |
| | 2.5, 3.5 and 4.5 mph | |
| 4 | Ascend/Descend stairs - 6 of each | 10 min |
| 5 | Sit/Stand (with fidgeting) | 12 min (6 min each) |
| 6 | Walking: 6 minute trials, 5 minute rest | 33 min |
| | between trials | |
| | Graded trials: 2.5 mph at ±1.5% grade | |
| | Loaded trial carrying 10% of body weight | |
| 7 | Cycling: 75 rpm and 50 rpm | 10 min (5 min each) |
| | Total: | 141 min |

3. Integrity Review

The data collected during the study were manually reviewed for integrity through a software package written in Labview. The review revealed that in 7 subjects the solder connections on one or more pressure sensors failed due to the pressures under the feet. The common mode of failure was break in the trace on the flexible insole resulting in a flat line output for all activities. The remaining 9 subjects had no failures in the sensor data and are referred further as the ‘no failures’ group.

B. Methods

1. Preprocessing of the Data

Only minimal preprocessing consisting of feature vector forming and normalization was applied to the sensor data. No other features were extracted. The feature vectors were formed to represent a time period (epoch) of two seconds in duration. Time histories of pressure and acceleration from both shoes were used as follows. A single sample of data from a shoe is represented by vector S={A_{AP}, A_{ML}, A_{SI}, P_{H}, P_{MO}, P_{MM}, P_{MI}, P_{T}}, where A_{AP} is anterior-posterior acceleration, A_{ML} is medial-lateral acceleration, A_{SI} is superior-inferior acceleration, P_{H} is heel pressure, P_{MO}, P_{MM}, P_{MI} are pressures from outer, middle and inner metatarsal sensors, respectively, and P_{T} is pressure from the big toe sensor. The time series of data from both shoes can be represented as fₜ₌{S_{L}, S_{R}}ₜ, i={1, . . . , M}, where Sₗ,S_{R} are the data samples from the left and right shoe, respectively, and M is the length of time series. The feature vector for an epoch e was produced using a decimation factor d as F_{e,d}={f_{e*N+d*k+1}}_{k∈{0, . . . ,[N-1/d]}}, where N is the number of samples in an epoch at the original 25 Hz sampling frequency (N=50 equivalent to a 2 second epoch used in this study), and k is the selection index. Use of decimation is equivalent to resampling of the original signals to a lower frequency. For example, d=1 corresponds to a sampling frequency of 25 Hz, d=5 corresponds to 5 Hz, etc. allowing to study the effects of changes in the sampling frequency on recognition accuracy. Due to decimation, the size of the feature vectors with all sensors included may vary from 800 elements (d=1, 2 shoes×8 sensors×25 samples per second×2 seconds=800 samples) to 32 elements (d=25, 2 shoes×8 sensors ×1 samples per second×2 seconds=32 samples). The features vectors from all epochs in the experiment were combined in a feature matrix F̅_{e,d} and all columns of the matrix were normalized to the scale of [0,1].

2. Classification by SVM

The pairs of feature vectors and class labels {F_{e,d},Lₑ} were presented to a supervised classification algorithm for training and validation. The labels Lₑ represented a distinct class {1-sitting, 2-standing, 3-walking, 4-ascending stairs, 5-descending stairs, 6-cycling}. The selected classifier was a variation of Support Vector Machine (SVM) implemented as a Matlab package (libSVM). The choice of the classifier was defined by the consideration of the generalization ability. The maximum margin classifier implemented by an SVM is less prone to overfitting compared to other available methods. For the target application of automatic classification of postures and activities the ability to generalize effectively is extremely important. As an example, motion of the lower extremities during ambulation is not perfectly repeatable. Similar variation in sensor data is expected from other postures and activities. In addition, some of the recorded data segments may contain transitions between similar postures and activities introducing the data, which cannot be perfectly labeled as one the classes. Thus the classifier is posed with a difficult task of learning a decision boundary, which should provide the best generalization from expectedly imperfect data.

The SVM classifier utilized Gaussian kernel (exp(−γ* (u−v)²). The best values of parameter C=10 (cost of misclassification) and y=0.0156 (width of Gaussian kernel) were found in grid search procedure varying C as C=10^{x}, x={−1, . . . ,3} and y as y=2^{y}, y={−8, . . . ,−2}.

3. Training, Validation and Calculations of Accuracy

A common training and validation procedure was deployed for all analyses. Specifically, a 4-fold cross validation was utilized where three quarters of all data were used as training set and the remaining quarter was used as validation set. The accuracy was reported as an average across 4 folds.

4. Six-Class Individual Models

The individual models are the best fit to the individual traits and thus represent the baseline accuracy for comparison. For the individual models, the folds were computed for each subject. All postures and activities were proportionally represented in each of the folds. All sensors were utilized in feature computation and dwas set to 1.

5. Six-Class Group Models

These models established group classification accuracies in the whole population of subjects as well as in the smaller ‘no failures’ group. The goal was comparison of recognition accuracies between these two group and the individual models, and evaluation of impact of solder connection failures. For these and other group models, the folds were organized by including the full dataset from each individual subject that belonged to a fold. All shoe sensors were utilized, and the decimation factor dwas set to 1.

6. Investigation of Best Sensor Configuration on the Group of 9 Subjects with No Sensor Failures

The goal of this analysis was to investigate the contribution of each individual sensor to recognition accuracy and determine the best sensor configuration. The study was performed using the backward selection procedure. First, the baseline population-average accuracy was established in configuration with all 8 sensors active. Next, sensors were excluded one at a time from the base configuration and accuracy of recognition was evaluated by a 4-fold cross-validation. On the next step, the configuration with highest accuracy from the previous step became the base configuration. The procedure was repeated until only one sensor was left. Since excluding a failed sensor could boost the recognition accuracy and lead to incorrect interpretation of sensor configuration, this procedure was performed only on the group of 9 subjects with no solder connection failures. The testing was performed with a decimation factor d=1.

7. Recognition Sensitivity to Sampling Frequency

Even momentary snapshots of pressure and acceleration are very unique to a given posture or activity. This analysis tested the hypothesis that using combination of pressure and acceleration performs well even at a lower sampling frequency. The accuracy of pattern classification was established on the group of 9 ‘no failures’ subjects using the best known sensor configuration and a range of decimations d={1,2,3,4,5,6,7,8,9,10,16,20,25} equivalent to the range of sampling frequencies of 25 to 1 Hz.

8. One Shoe vs. Two Shoes

The effect of wearing one sensor-equipped shoe vs. two shoes was investigated by changing the way the feature vectors were formed. Specifically, the feature vector was formed either as fₜ={S_{L}}ₜ, or fₜ={S_{R}}t. Thus recognition accuracy from the data generated only by the left or right shoe can be compared to the data acquired from both shoes.

C. Results

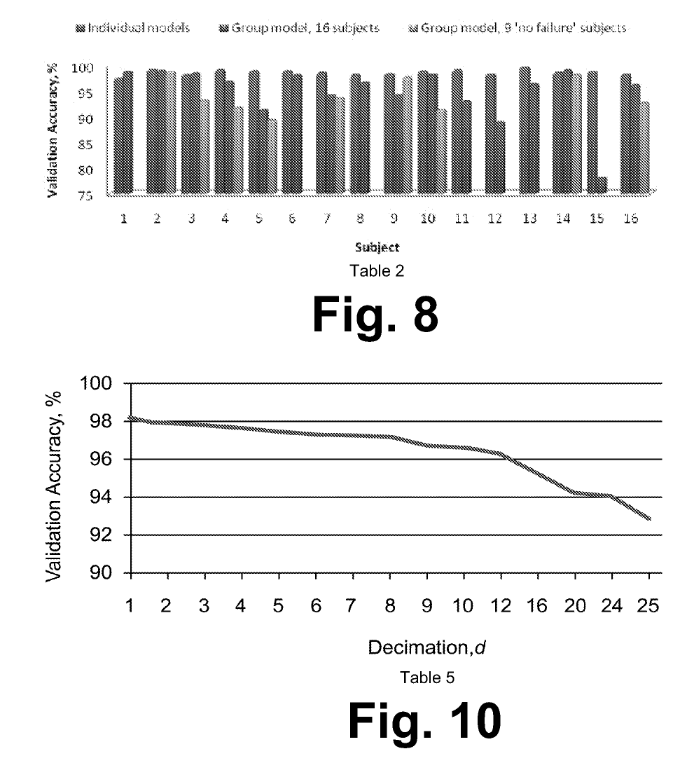

Table 2 shown in FIG. 8 illustrates 6-class average validation accuracy obtained by individual, and 16- and 9-subject group models for each subject. The population average accuracy obtained by the individual models was 98.6±0.5%; the 16-subject group model 94.9±5.2%; and the 9-subject ‘no failures’ group 94.1±3.1%.

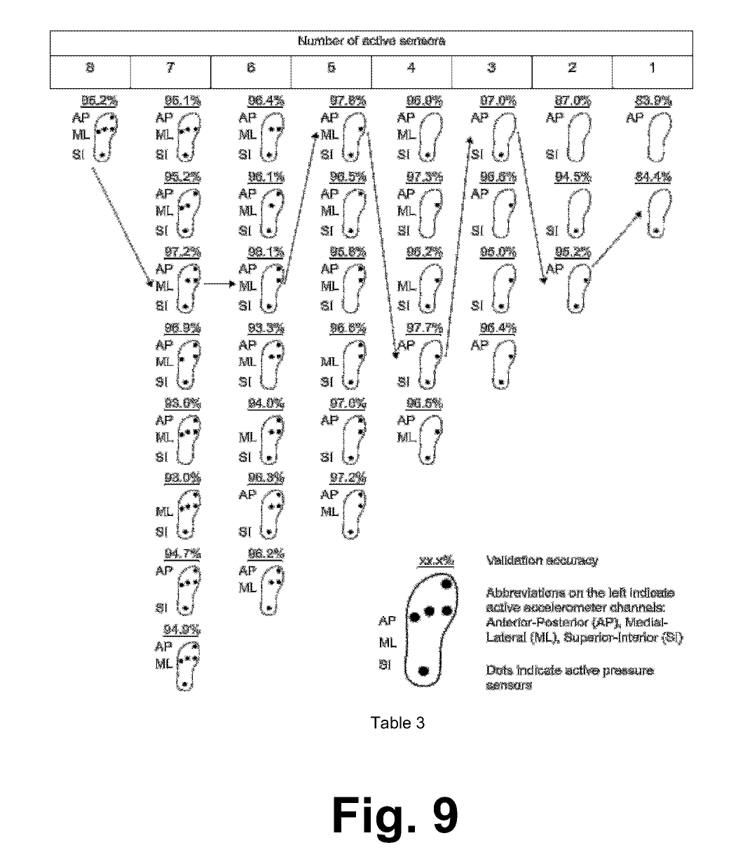

The results of the backward selection of various sensor configurations are shown in Table 3, which is presented in FIG. 9. The first bar in each grouping of bars represents individual models; the second bar in each grouping of bars represents a group model of 16 subjects; and the third bar in each grouping of bars represents a group model of 9 “no failure” subjects. The highest population-average recognition accuracy of 98.1% is achieved in a configuration with {PH,PMI,PT, AAP, AML, ASI} sensors.

The population-cumulative confusion matrix for recognition using the best sensor configuration is presented in Table 4:

TABLE 4

| | Predicted class | | | | | | |
|---|---|---|---|---|---|---|---|
| | | | | | | | Class- |
| | | | | | | | specific |
| | Sit | Stand | Walk | Ascend | Descend | Cycle | recall |
| Actual Class | | | | | | | |
| Sit | 3202 | 2 | 0 | 0 | 0 | 14 | 0.99 |
| Stand | 7 | 3191 | 2 | 7 | 0 | 0 | 0.99 |
| Walk | 0 | 0 | 10647 | 74 | 0 | 0 | 0.99 |
| Ascend | 0 | 0 | 34 | 500 | 15 | 1 | 0.89 |
| Descend | 0 | 0 | 41 | 60 | 405 | 0 | 0.80 |
| Cycle | 146 | 3 | 0 | 0 | 0 | 2539 | 0.96 |
| Class- | 0.97 | 1.00 | 0.99 | 0.82 | 0.96 | 0.99 | 0.98 |
| specific | | | | | | | |
| precision | | | | | | | |

The graph of the recognition accuracy as a function of the decimation factor is shown in Table 5, which is presented in FIG. 10. The population-average accuracy of recognition in configuration with one shoe only was 95.9% for the left shoe and 94% for the right shoe.

D. Discussion

The proposed device achieved the greater recognition rates (95%-98%) compared to previous experiments that used similar postures and activities. For example, demonstrated 88% percent accuracy with 6 postures and activities. The proposed shoe-based approach also matched or outperformed other single-location methodologies such as which reported a 95% accuracy across 8 postures and activities. The proposed device should be capable of maintaining the accuracy as other metabolically significant activities are classified (e.g., elliptical trainer)).

The device has shown excellent accuracy using group models, suggesting that individual calibration is not necessary. As Table 2 shows, the 98.6% average accuracy of the individual models is similar to the 98.1% accuracy of the group model for 9 ‘no failure’ subjects. A comparison to the full 16-subject group model shows a small 3% decrease in accuracy due to effects of sensor failures for some subjects. Sensor failures also explain the increased variance of the results. Subject number 15 suffered from multiple sensor failures at the very beginning of data collection and is an obvious outlier. This subject is a good example that demonstrates the effects of failed sensors: the individual model shows high recognition accuracy because of the redundancy in the pressure readings and the 16-subject group model shows a substantial reduction of accuracy because the sensors normally present in other subjects have failed in this subject.

As the confusion matrix in Table 3 shows, the shoe device achieves greater than 80% precision and recall in recognition of difficult activities such as ascending and descending stairs. The actual accuracy may actually be even higher as the subjects had to take several steps on a flat surface (which is correctly is classified as walking) when transitioning from one flight of steps to another.

The backward selection of the best sensor configuration shown in Table 2 clearly illustrates the redundancy in the sensor data. The best population-average accuracy of 98.1% is achieved by discarding signals from P_{MO} and P_{MM}. It is possible that the pressure patterns on those sensors may carry more individual traits than P_{MI}. This figure also demonstrates the complementary nature of plantar pressure and heel acceleration patterns. While the population-average recognition accuracy using just AP acceleration is 83.9% and using just heel pressure is 84.4%, combining these two together provides an immediate increase of over 10% to 95.2%. High accuracy in configurations with only left and right shoe indicates that only one shoe can be equipped with sensors and still maintain high accuracy of classification.

Table 4 effectively demonstrates tolerance of the proposed combination of sensor modalities to lower sampling frequencies. While the highest accuracy of 98.1% is observed at 25 Hz, the relative decline for 5 Hz sampling is only 0.6% (accuracy of 97.5%). As example, a relative decline of 12% (from 85% to 75%) was reported while changing sampling from 25 Hz to 5 Hz for Y axis of an accelerometer. This useful property allows for lower data rates in a body network and a potential for extended battery life.

Similarly, the proposed methodology does not need signal processing and feature extraction beyond simple forming of vectors and normalization. This compares very favorably with extensively used frequency domain features that need substantial computing power and thus may present a heavy burden for a wearable computing platform.

Finally, it is possible to substantially increase durability of the pressure sensitive insole by changes in the manufacturing process. The reason for failures in 7 subjects was not the pressure sensors themselves but rather high pressure points created by solder connections. These points failed under substantial impact forces of walking and jogging. Eliminating the solder connections or encapsulating them into elastic buffer material should resolve this issue.

### II. Second Experiment (Using a Capacitive Sensor)

Research was focused on the design of a novel pressure-sensitive sensor that would utilize the sole of person's foot as one of the capacitor plates. Such a design offered much higher durability, cost savings and allow incorporation of truly novel functionality for weight measurement. Body weight is a metabolically-relevant physiological indicator that can further improve the accuracy of the device.

The research was carried out as a series of Tasks, each with its own deliverable:
Task 1—Develop a novel capacitive sensor where one plate is the person's foot. Practically establish the range of capacitances for the sensor as a function of plate topology.
Task 2—Find the plate topology with the lowest Equivalent Series Resistance.
Task 3—Characterize the capacitive sensor in static loading tests.
Task 4—Implement capacitive measurement methodology using the MSP430 microcontroller used in the existing activity monitor.

To enable weight measurement the tested sensor spanned the whole area of the insole. Thus, changes in the distribution of weight on the sole of foot did not change the measurement. Use of a single pressure sensor was different from the existing shoe prototype, but research has shown that one pressure sensor is sufficient for highly accurate posture and activity recognition. A thin (8 mil) flexible insole had two isolated conductive plates (shown as green and blue areas) interleaved in a comb-like structure. The interleaving minimized Equivalent Series Resistance (ESR) in the biological tissue of the foot. Pressure applied to the top plate changed the gap d between the plates. Higher pressure resulted in a smaller gap and higher capacitance. The equivalent electrical circuit consisted of two variable capacitors in series.

To characterize the sensors, a series of experiments was conducted in which a realistic model of a foot was utilized. This eliminated the need for human subjects testing. The plastic hollow models of the foot was be acquired from an anatomy warehouse. These models represented high, normal arches and flat foot. To closely simulate properties of real living tissue, the models were be filled with a conductive gelatin solution which closely resembles electrical properties of the body. Weights were be added to the feet to apply a known amount of pressure to the sensor.

A. Task 1: Practically establish the range of capacitances for the sensor as a function of plate topology

The expected value of sensor capacitance was estimated based on the following considerations. The surface area of the insole varies approximately from 125 cm² (women's US size 5) to 250 cm² (men's US size 12). The capacitance in the simple plate model can be expressed as C=ε_{γ}εₒ A/d, where ε_{γ} is the relative static permittivity (dielectric constant) of the material between the plates,

$${ɛ}_{o} = 8.854 E - 12 \frac{F}{m}$$

is the permittivity of free space, A is the area of overlap between plates in m², and d is the distance between plates in meters. The estimate of C1 and C2 values can be obtained under following assumptions: 1) C1 and C2 represent approximately half of the surface area each 2) C1≈C2 3) ε_{γ}≈3.5 is that of rubber foam with approximately 50/50 air to rubber volume ratio, 4) typical thickness of the foam padding separating the foot and the capacitive plates is d=3 mm uncompressed and d=1 mm compressed. Then, for women's size 5, the minimal capacitance C↓1↑MIN=3.5*8.854E−12*0.0125/(3E−3)=129 pF. Similarly, the maximum capacitance in men shoe size 12 is C↓1↑MAX=3.5*8.854E−12*0.025/(1E−3)=774 pF. Thus, the expected range of capacitances for C1 and C2 is from 129 pF to 774 pF. Under ideal conditions, the capacitance of the sensor is equivalent to the capacitance of the series connection:

$${C}_{sensor}^{MIN} = \frac{{( {C}_{1}^{MIN} )}^{2}}{2 {C}_{1}^{MIN}} = 64.5 pF and {C}_{sensor}^{MAX} = \frac{{( {C}_{1}^{MAX} )}^{2}}{2 {C}_{1}^{MAX}} = 387 pF .$$

Thus expected values of capacitance were very close to those typically used in capacitive proximity and pressure sensors. However, the calculations above were based on a number of idealized assumptions. In practice, values of C1 and C2 depended on the complex interaction of the shape of the footprint and weight distribution which is hard to evaluate analytically. To identify the best possible plate topology on the flex insole, two plate configurations were tested: 1) uniformly distributed comb, and 2) comb following the pressure pattern in standing. The insoles with such patterns were fabricated using photo transfer and chemical etching process on flexible PCB material from LPKF. A copper electrode was inserted into the artificial foot and will act as the middle electrode. Capacitance of C1 and C2 will be practically measured in relation the middle electrode under pressures of 400-4000 Pa. The result was a configuration of plates that provides equivalent changes in C1 and C2 under load corresponding to standing (position in which the weight measurement will be taken).

B. Task 2: Establish the Plate Topology with the Lowest ESR

The plate topology was initially analyzed in Task 1 to ensure approximately equal values of C1 and C2. The second goal was to look at the effect of plate topology on the ESR. While the step of the comb structure spacing has no bearing on capacity (the area of overlap remains constant), it may have a significant effect on ESR. Indeed, the ESR was reduced as the length of the line separating two plates' increased (which in turn involves more tissue into equivalent electrical contact). A step size from the set {2 cm, 1 cm, 0.5 cm, 0.25 cm} was tested. The insoles with the varying comb structures were fabricated using photo transfer and chemical etching process. The ESR of the sensor was measured using AnaTek ESR meter under various loads. As the result of Tasks 1 and Task 2, the optimal plate topology was established.

C. Task 3: Characterize the Capacitive Sensor in Static Loading Tests

Sensitivity, non-linearity, repeatability and hysteresis were important parameters defining the basic accuracy of the sensor and were needed for additional numerical correction (e.g. non-linearity) or statistical processing of the measurements.

This experiment utilized an artificial prosthetic foot capable of carrying loads in excess of 100 kg, e.g. Flex-Foot Axia by Ossur. The loading characteristic of the sensor (capacitance vs. applied weight) was constructed using a set of weights in the range of 5-100 kg applied through the prosthetic. The weights were progressively loaded and unloaded from the foot. The resulting loading curve was used to calculate the following standard characteristics: sensitivity (pF/kg), repeatability (%), non-linearity (%), and hysteresis (%). These values determined the need for additional numerical correction of the sensor output for practical weight measurement.

D. Task 4: Demonstrate Continuous Capacitive Sensing by an Inexpensive Microcontroller-Based Circuit

This task had two goals: first, a design of software and hardware for continuous real-time (at least 25 Hz update rate) monitoring of sensor capacitance; second, proof of commercial viability of the proposed sensor which allow substantial saving to the cost of FSR.

The capacitive sensing was performed by a MSP430 microcontroller which was already incorporated into the shoe electronics. The principle of operation was be based on measuring discharge time of an RC circuit in which the capacitor is the pressure sensor. A general-purpose pin in output mode charged the capacitor to a known voltage. Then a timer was started and the pin was switched to input mode. The capacitors discharged though a known resistance R. When the voltage on the capacitor crossed the low threshold voltage of the input pin, an internal interrupt was generated which stopped counting of the internal timer. The captured number of Timer clicks (discharge time) was proportional to the capacitance C. The capacitance C was in the range of between 64.5-387pF. The discharge time in an RC circuit to near ground was approximately T_{DISCHARGE}≈5t≈5RC. Choosing R value to be 1M, the discharge time varied between 322 uS to 1.9 mS, corresponding to sampling frequencies better than 500 Hz. In input configuration, the MSP430 microcontroller had a ±50 nA leakage port current which was negligible compared to the discharge current through resistance R (3 uA at 3V) and thus did not impact the accuracy. The value of the pressure sensor's ESR was taken into consideration if necessary (i.e. if it was high enough to influence discharge time). A 16-bit Timer A was clocked using 16 MHz crystal, which resulted in 5000 to 30400 counts per each measurement (from min capacitance to max capacitance). Resulting discretization of the capacitance was fine enough to capture even minute variations in the weight.

This Task was started in parallel with Task 1 as they were independent. The output of capacity measurement was visualized through a serial connection from the MSP430 development board to a personal computer. At the end of the design phase we were able to capture pressure readings in real time from a person wearing the shoes. The resulting design cost pennies compared to an expensive (several dollars) FSR used in the current design and was considerably more durable (durability will be tested after design-for-manufacture in Phase II). Ultimately this contributed to better affordability of the shoe monitor.

III. Third Experiment (Using Force Sensitive Resistor Pressure Sensors)

A. Shoe Design

Data for the pilot study was collected using a prototype pair of instrumented shoes. The insole of each shoe was equipped with 5 Force-Sensitive Resistors (FSRs). The FSRs were located under the heel, metatarsal bones and the toe. A three-dimensional MEMS accelerometer (LIS3D02AQ) was mounted on the heel of the shoe. Pressure and acceleration data were sampled at 25 Hz and sent over a wireless link to the base computer. The battery, power switch and the wireless board are all installed at the back of the shoe. The whole design was very lightweight and created no interference with normal motion patterns. It should also be noted that this design was very inexpensive (<$100 in mass quantities) and durable.

B. Data Flow in the System for Weight and Energy Expenditure Measurement

Acceleration sensors and/or pressure sensors were incorporated into the shoe or implemented as a shoe insert (insole). A physiological sensor may measure heart or respiration rate. Examples of the physiological sensor are: piezoelectric pulse monitor located on a wrist or an ankle or inside of the shoe system; reflectance optical oximeter detecting oxygenation and/or pulse located on a wrist or an ankle or inside the shoe system; respiration sensor (a plethysmographer) located around the chest. The physiological sensor may have a wired or a wireless connection. The physiological sensor is optional and may be used for higher accuracy of measuring metabolic activity.

Data from the sensors (acceleration, pressure, and optional physiological sensor) was delivered by a wired or a wireless connection to a data processing device. The data processing device may be a dedicated device (i.e. a wrist unit that could also be combined with a physiological sensor, or a personal computer) or a ubiquitous computing device such as a cell phone or PDA. The data processing device applied methods of signal processing such a filtering, normalization and others to condition the sensor signal for further processing. Then the continuous signals were split into short segments (epochs) for which prediction will be made and features of interest were extracted (see Table 5 for an example) such as time-lagged measurements of pressure and acceleration, and/or energy measures (RMS, etc.), and/or entropy measures and/or time-frequency decompositions (short-time Fourier transform, wavelets, etc). The features were representative of the posture/activity and intensity with which a posture/activity is performed. The features characteristic of posture and activity were fed into a classifier that performs recognition of the posture/activity. For example the classifier can be implemented as an artificial neural network such as LIRA (Appendix A), Multi-Layer perceptron or other network. Alternatively the classifier may be a machine learning algorithm such as a linear or non-linear discriminant, parametric or non-parametric model, etc. For example, classifications can be performed by Support Vector Machines or other methods. Features characteristic of intensity of posture/activity were fed into a regression model defined specifically for each posture/activity. The regression model took features and parameters (for example, weight of the person) as inputs and produces estimates of energy expenditure as the output. This output can be summarized in a number of ways (total calories burned, calories per posture/activity, calories above/below the target, daily trends, weeks/monthly trends, etc) and presented as biofeedback to the user. The device can also detect prolonged periods of low activity and cue the user on performing physical exercise.

C. Data Preprocessing

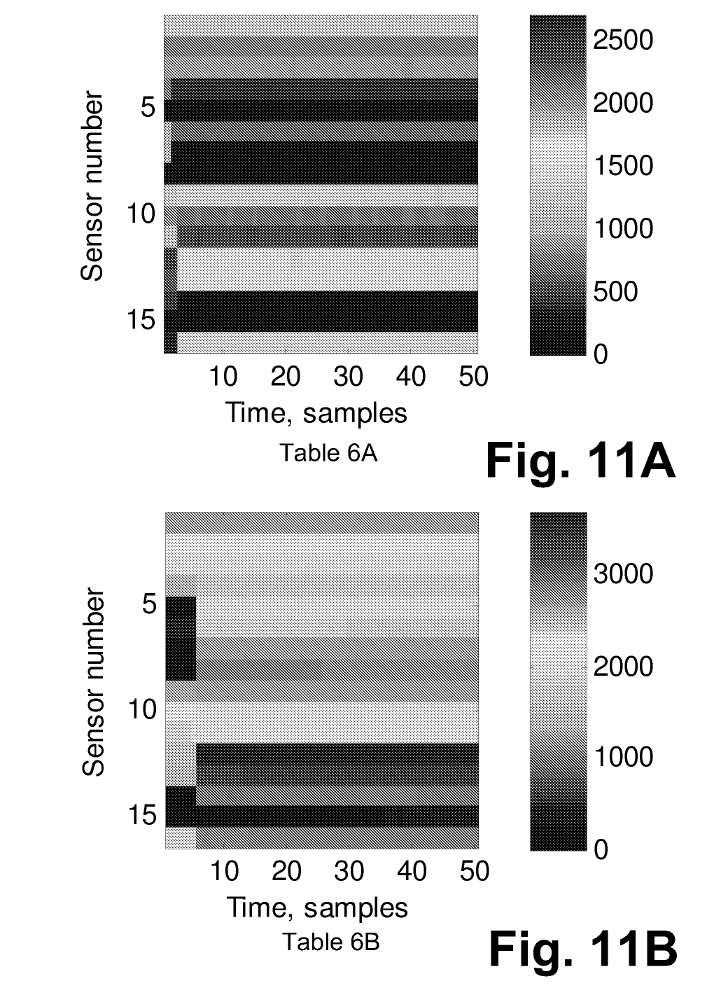

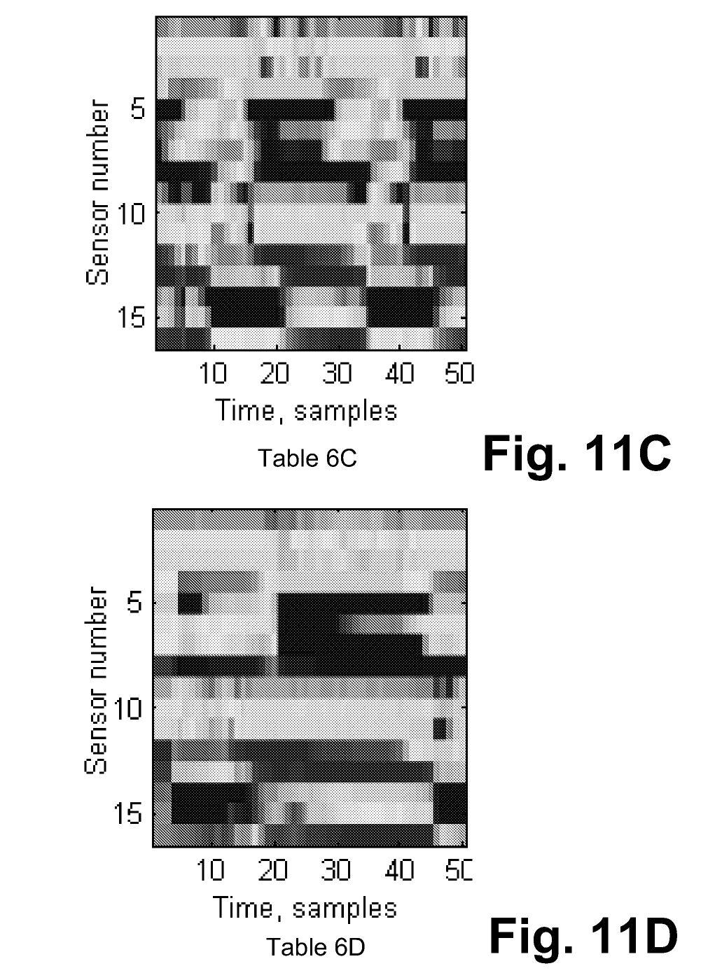

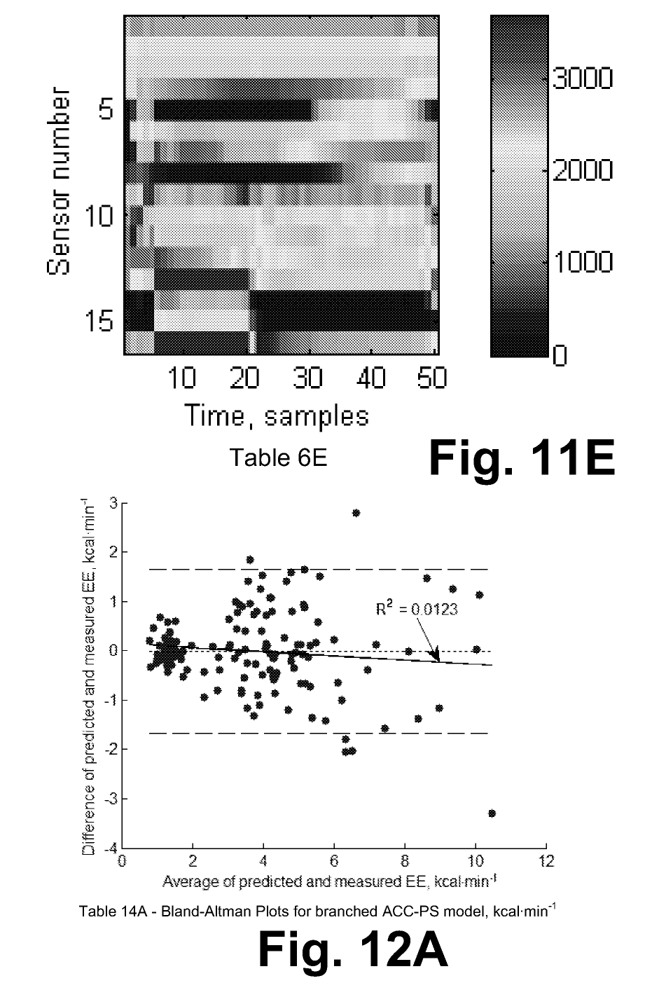

Captured sensor data was processed to form feature vectors for the classifier. Each 800-element feature vector represents pressure and acceleration histories from both shoes for the past two seconds (2 shoes×8 sensors×25 samples per second×2 seconds=800 samples). Thus, all predictions were made for non-overlapping 2-second epochs. Tables 6A-6E show a two-dimensional representations of the feature vectors for each posture/activity. The X-axis shows time progression and Y-axis shows color-coded reading from the sensors in ADC units. First 8 sensors (top half of each image) correspond to the left shoe and next 8 sensors (bottom half of each image) correspond to the right shoe. As Tables 6A-6E presented in FIGS. 11A-11E show, each posture and activity creates distinct features that can be used by the classifier.

D. Classifier Training and Validation

Twenty-five percent (25%) of the collected dataset was used for training and 75% for validation (reporting of the results). Each posture/activity was represented in the same proportion both in training and validation sets.

Each feature vector was assigned a label representing a distinct class (1-sitting, 2-standing, 3-walking, 4-ascending stairs, 5-descending stairs). The feature vectors and corresponding labels from the training set were presented to a multi-class Support Vector Machine (SVM). SVM is known for robust theoretical foundation and generalization capabilities. Data from the training set were used to train a classifier that would assign a label (1-5) to a presented feature vector.

Finally, the data from the validation set were presented to the classifier. Predicted labels were compared against expected. Multiple experiments were conducted in which the content of training and validation sets was randomly selected from available data. The accuracy of prediction varied from 98% to 100% for multiple randomized trials. These results demonstrate that the proposed device is capable of accurate recognition of a variety of postures and activities.

### IV. Fourth Experiment

The goals of this study were: 1) to show the improvement in the accuracy of energy expenditure prediction using shoe-based device over existing methods in the area; 2) to demonstrate the superiority of prediction performance of model using accelerometer and pressure sensors signals over the models that use only accelerometer signals obtained from the wearable shoe sensors; 3) to validate the branched modeling approach for prediction of energy expenditure using shoe-based device; 4) to establish the need of sensors to be embedded in both shoes.

A. Subjects

Sixteen subjects (8 males and 8 females, 18-44 yr, 48.6-119.8 kg, 61-72 in., 18.1-39.4 kg/m2) were included in the study. They were asked to perform the a variety of activities while wearing shoes with sensors. All subjects were healthy with a mean peak O₂ uptake (Vo₂) of 23.2 ml·min⁻¹·kg⁻¹ (range: 15.18-33.35 ml·min⁻¹·kg⁻¹). Informed, written consent was obtained from each participant before entering the study.

B. EE Measurement

Energy expenditure for each 1-min. activity was measured by indirect calorimetry. In indirect calorimetry the measurements of respiratory gases (oxygen uptake and carbon dioxide production) are used to predict the total amount of oxygen consumed, which in turn is used to estimate energy expenditure. Oxygen uptake and carbon dioxide production was measured during each activity, using a portable gas analysis system. Energy expenditure (kcal·min⁻¹) was converted from predicted oxygen consumption using equivalence of 1 liter of consumed oxygen to 4.78 kcal of energy expended.

C. Movement and Foot Pressure Measurement.

The sensor data for this study was collected by a wearable sensor system embedded into shoes. Each shoe incorporated five force-sensitive sensors embedded in a flexible insole and positioned under the critical points of contact: heel, metatarsal bones and the toe. Such positioning allowed for differentiation of the most critical parts of the gait cycle such as heel strike, stance phase and toe-off. The information from the pressure sensors was supplemented by the data from a 3-dimensional accelerometer positioned on the back of the shoe. The goal of accelerometer was to detect orientation of the shoe in respect to gravity, to characterize the motion trajectory and to help characterize a specific posture or activity (for example, ambulation velocity). Pressure and acceleration data were sampled at 25 Hz and sent over a wireless link to the base computer. The wireless system used for data acquisition was based on Wireless Intelligent Sensor and Actuator Network (WISAN) developed specifically for time-synchronous monitoring applications. Application of WISAN allowed for data sampling at exactly the same time from both shoes, thus avoiding potential complications that could be created in systems with varying time delay between sensors. The battery, power switch and the WISAN board were installed at the back of the shoe. The sensor system was very lightweight and created no interference with motion patterns in subjects.

D. Study Protocol

Each subject was asked to perform a variety of 1-min. activities while wearing the gas mask and the appropriately sized shoe device with embedded sensors. There were 13 different activities from four groups (Sit, Stand, Walk and Cycle) performed by each subject, see Table 7.

TABLE 7
Four groups of activities performed by subjects according to protocol

| Sit | Stand | Walk | Cycle |
|---|---|---|---|
| Sit motionless | Stand motionless | Walk 1.5 mph | Cycling 50 rpm |
| Sit fidgeting | Stand fidgeting | Walk 2.5 mph | Cycling 75 rpm |
| | | Walk 3.5 mph | |
| | | Jog 4.5 mph | |
| | | Walk downhill | |
| | | Walk uphill | |
| | | Walk loaded | |

The data consisted of 1-min experiments (1 for each activity, for the total of 13 activities) obtained from every subject. Thus, for the 16 subjects that originally participated in the study there were 208 1-min experiments.

The following data were available for every experiment:
response variable: energy expenditure, EE, kcal·min⁻¹;
anthropological measurements (weight, height, BMI, age, gender, shoe size)
triaxial accelerometer readings obtained for a 1-min period for a specific activity performed by every subject (3 signals: superior-inferior (Acc1), medial-lateral (Acc2), anterior-posterior(Acc3), each of length approximately 1,500 data points): and
pressure sensor readings obtained simultaneously with accelerometer readings for a 1-min period for a specific activity performed by every subject (5 signals: heel (Sens1), middle meta (Sens2), left meta (Sens3), right meta (Sens 4), toe (Sens5), each of length approximately 1,500).

Four models were constructed to predict EE in kcal·min⁻¹ using this data. These consisted of two models branched by activity (“Sit”, “Stand”, “Walk”, “Cycle”): branched ACC-PS (included physical measurements, accelerometer and pressure sensors predictors) and branched ACC (included physical measurements and accelerometer predictors), and also two nonbranched models with sets of predictors corresponding to the branched versions: nonbranched ACC-PS and nonbranched ACC. The purpose of constructing the models was to investigate if the performance was improved by branching the model and also by including pressure sensor predictors.

Accelerometer and pressure sensors signals were preprocessed to extract meaningful metrics to be used as predictors for the model. For each sensor, the following metrics were tested for the inclusion into each model as predictors: coefficient of variation (cv); standard deviation (std); coefficient of variation (cv); frequency which is computed as the number of “zero crossings,” i.e. the number of times the signal crosses its median (frq) normalized by the signal's length; entropy Hof the distribution X of signal values (ent) computed as:

H(X)=−Σpₖ log pₖ.

For each model, the derived metrics were used as possible predictors for the ordinary least squares (OLS) linear regression. The transformed predictors (log, inverse and square root) and interactions (as products of 2 or more candidate predictors) were also considered as separate linear terms within regression.

In branched models, a separate branch model was constructed for each identified posture activity: “Sit”, “Stand”, “Walk” and “Cycle”. For each model (branch activity models and nonbranched models), selection of the most significant set of predictors was performed using the forward selection procedure. We used the “leave-one-out” approach for cross-validation when training and predicting the EE for each experiment for every subject. Within each model there were several activities performed by each subject, all of such experiments related to the same subject were removed from the training set. A model (coefficients) computed using the rest of the subjects was then used to predict the EE for all experiments of the left out subject. The best set of predictors had to provide the best fit (by producing the maximum adjusted coefficient of determination, R²_{adj} and the minimum Akaike Information Criterion, AIC) in the training step and the best predictive performance (the minimum mean squared error, MSE and the minimum mean absolute error, MAE) in the verification step.

Originally, the study included 16 subjects. As a result of data quality analysis, it was detected that subjects 6, 8, 11, 12, 13 had pressure sensors failure on both shoes in at least one activity group experiments and, therefore, they were completely excluded from the analysis. Thus, the input for the model was the set of 1-min experiments from 11 subjects (4 males and 7 females). The summary statistics of the physical characteristics of the 11 subjects used for subsequent model construction are shown in Table 2. In the “walk” activity group, some subjects did not have an energy expenditure record or had no sensors recorded for some experiments within this group. These 4 experiments were dropped from each model's input. Thus, the sample size of the input data for each model was (11×13)−4=139 experiments.

TABLE 8
Summary statistics for anthropometric characteristics
of 11 subjects used for model construction

| | | Standard | | | |
|---|---|---|---|---|---|
| Characteristic | Mean | deviation | Median | Min | Max |
| Age, years | 25.8 | 7.2 | 24 | 18 | 44 |
| Weight, kg | 71.14 | 14.34 | 69.6 | 55 | 100.9 |
| BMI | 25.58 | 6.07 | 24.06 | 18.73 | 39.41 |
| Height, inches | 65.93 | 3.44 | 66 | 61 | 71 |
| ShoeSize | 8.68 | 1.23 | 8.5 | 7 | 10.5 |

Measured and predicted energy expenditure values in kcal·min⁻¹ for each experiment were then converted to METs (kcal·kg⁻¹·hour⁻¹) for both branched ACC-PS and ACC models and their nonbranched versions.

One of the goals of the analysis was to establish the need of using sensors on both shoes. Preliminary analysis showed that metrics derived from the difference between the signals from left and right shoes exhibited good predictive power for the model. Namely, the frequency (i.e. the number of “zero crossings”) of the difference signal between left and right shoe sensors for pressure sensor 4 showed improvement if included into the “Walk” branch model. Several versions of the branched ACC-PS model (as a representative model) were constructed using accelerometer and pressure sensors data separately from each shoe and both shoes together.

E. Statistics

The following performance assessment measures were computed for each model predicting energy expenditure per experiment in kcal·min⁻¹ or METs):

RMSE_{MET}—the root mean squared error for energy expenditure prediction expressed in METs. This error is computed as the difference between model predicted EE and the measured EE for each experiment.

ARD—the Average Relative Difference (signed):

ARD=mean((predEE−EE)/EE)

AARD—the Average Absolute Relative Difference:

AARD=mean(|predEE−EE|/EE)

RMSE_{%}—the RMSE expressed as the percentage of the mean measured energy expenditure (in METs)

Bias—the mean difference between predicted and measured energy expenditure in METs:

bias=mean(predEE−EE)

Interval of agreement—a prediction of energy expenditure in METs: (bias±2·SD(bias))

A Bland-Altman plot analysis was conducted to reveal any systematic pattern of the error (calculated as the difference between predicted and measured EE) across the range of measurements (as the mean of predicted and measured EE) and to assess the bias and interval of agreement for prediction of EE.

Passing-Bablok regressions (as a robust alternative to least squares regression) for all four models and for two units of prediction (kcal·min−1 and METs) were constructed. Passing-Bablok regression is best suited for method comparison because it allows measurement error in both variables, does not require normality of errors and is robust against outliers. In addition, Passing-Bablok regression procedure estimates systematic errors in form of fixed (by testing if 95% CI includes 0) and proportional bias (by testing if 95% CI includes 1).

F. Results

First, the effect of inclusion of predictors was investigated from both shoes into the model using branched ACC-PS model as an example.

Table 9 below shows comparative performance of the models that used the best selected set of predictors (cv, std, frq and ent, computed separately for each shoe) and the difference metrics derived from the difference between signal form left and right shoe. Each model's performance is reported as the aggregated results from four branch models (“Sit”, “Stand”, “Walk” and “Cycle”). “Mean” and “Max” models used respectively mean and maximum values of all predictors (accelerometer and pressure sensors), “Left” and “Right” models used only signals from left or right shoe. The “difference” model included the difference metric in addition to the previously selected set of predictors. As Table 3 indicates, there was almost no improvement provided by inclusion of the difference metrics when compared to the rest of the models. Overall, models based on metrics derived for both shoes (“Mean”, “Max” and “Difference”) performed slightly better than single shoe models. However, this improvement is not significant and for all practical purposes single shoe models can be successfully used instead. In addition, it should be noted that in the data set some subjects had pressure sensor failure on one of the shoes (resulting in derived metrics being close to 0). This loss of information can explain poor results for single shoe models reported in Table 9. On the other hand, such failures did not particularly affect either “Mean” or “Max” since it was accounted for during either averaging or, especially, maximum value selection.

TABLE 9
Comparison of model performance using predictors from single shoe and both shoes

| | | | | | | Interval of |
|---|---|---|---|---|---|---|
| | | | | | Bias, | agreement, |
| Predictors | RMSE_{MET} | ARD, % | AARD, % | RMSE, % | METs | METs |
| Mean | 0.6671 | 0.0295 | 0.1748 | 0.2092 | −0.0084 | (−1.3474, 1.3306) |
| Max | 0.6602 | 0.0291 | 0.1706 | 0.2070 | −0.0029 | (−1.3280, 1.3222) |
| Left | 0.6849 | 0.0318 | 0.1768 | 0.2147 | −0.0081 | (−1.3827, 1.3664) |
| Right | 0.6920 | 0.0316 | 0.1861 | 0.2170 | −0.0082 | (−1.3970, 1.3807) |
| Difference | 0.6666 | 0.0289 | 0.1698 | 0.2090 | −0.0019 | (−1.3398, 1.3361) |

The rest of the results reported here correspond to ACC-PS, ACC branched and nonbranched models constructed using mean of {left, right} accelerometer metrics and maximum of {left, right} pressure sensors metrics as an approximate “single shoe” model.

As a result of selection of the best set of predictors final branch models within branched ACC-PS and branched ACC models with included predictors and coefficients are reported in Table 10 and Table 11, respectively. Final nonbranched ACC-PS and nonbranched ACC models are given in Table 12. The coefficients for all models were obtained by averaging the coefficients of the 11 runs (one for each left out subject) of the OLS regression on the training sets. Almost all coefficients for all models were highly stable over all runs as given by low absolute values of coefficients of variation (CV). As can be expected, weight and BMI always explain part of the variability of each model, other physical characteristics were highly correlated to weight variable and didn't add to the fit or the prediction performance of either model.

TABLE 10
Regression coefficients for the ACC-PS model
(to predict EE in kcal/min)

| | | Average | |
|---|---|---|---|
| | | Values of | CV of |
| Branch Model | Predictors | Coefficients | Coefficients |
| Sit | <Intercept> | 4.3273 | 0.1518 |
| | Weight, kg | 0.0254 | 0.1779 |
| | log(BMI), ? | −1.3105 | −0.2204 |
| | log(Acc1.cv), ? | 0.1011 | 0.0345 |
| Stand | <Intercept> | 7.9942 | 0.1180 |
| | Weight | 0.0410 | 0.1175 |
| | log(BMI) | −2.2356 | 0.1449 |
| | log(Acc2.std) | 0.2168 | 0.0886 |
| | log(Sens4.std) | −0.1988 | −0.2063 |
| Walk | <Intercept> | 1.1857 | 0.8913 |
| | Weight | 0.0823 | 0.1050 |
| | log(BMI) | −2.3609 | −0.1843 |
| | Sens3.freq · Sens4.freq | 231.2702 | 0.1834 |
| | Acc3.std | −0.0005 | −0.5190 |
| | Acc3.ent · Acc2.ent · Acc1. | 0.4305 | 0.0763 |
| | ent | | |
| Cycle | <Intercept> | 6.5579 | 0.2960 |
| | Weight | 0.1056 | 0.1162 |
| | log(BMI) | −3.8577 | −0.1776 |
| | Acc1.std | 0.0041 | 0.0708 |
| | Sens5.std · Sens3.std | 0.0000086 | 0.0530 |

TABLE 11
Regression Coefficients for the ACC Model
(to predict EE in kcal/min)

| | | Average | |
|---|---|---|---|
| | | Values of | CV of |
| Branch Model | Predictors | Coefficients | Coefficients |
| Sit | <Intercept> | 4.3273 | 0.1518 |
| | Weight, kg | 0.0254 | 0.1779 |
| | log(BMI), ? | −1.3105 | −0.2204 |
| | log(Acc1.cv), ? | 0.1011 | 0.0345 |
| Stand | <Intercept> | 5.7161 | 0.1268 |
| | Weight | 0.0388 | 0.1315 |
| | log(BMI) | −1.9963 | −0.1647 |
| | log(Acc2.std) | 0.1161 | 0.0843 |
| Walk | <Intercept> | 1.6762 | 0.6302 |
| | Weight | 0.0872 | 0.0949 |
| | log(BMI) | −2.9632 | −0.1623 |
| | Acc3.std | 0.0004 | 0.5941 |
| | Acc3.ent · Acc2.ent · Acc1. | 0.6221 | 0.0264 |
| | ent | | |
| Cycle | <Intercept> | 10.0812 | 0.2688 |
| | Weight | 0.0859 | 0.1794 |
| | log(BMI) | −4.1971 | −0.2545 |
| | Acc1.std | 0.0048 | 0.0993 |

TABLE 12
Coefficients for nonbranched ACC-PS and ACC models

| | | Average values of | CV of |
|---|---|---|---|
| Model | Predictors | coefficients | coefficients |
| Nonbranched | <Intercept> | 2.8511 | 0.2497 |
| ACC-PS | Weight | 0.0644 | 0.1111 |
| | log(BMI) | −2.1651 | −0.1672 |
| | Acc1.ent · Acc1.ent | 0.3670 | 0.0703 |
| | Acc3.ent · Acc3.ent | 0.2217 | 0.1632 |
| | Acc2.std · Sens1.freq | 0.0372 | 0.0598 |
| Nonbranched | <Intercept> | 2.4845 | 0.2739 |
| ACC | Weight | 0.0628 | 0.1056 |
| | log(BMI) | −2.1002 | −0.1621 |
| | Acc1.ent · Acc1.ent | 0.3786 | 0.0811 |
| | Acc3.ent · Acc3.ent | 0.2575 | 0.1567 |
| | Acc2.std | 0.0052 | 0.0786 |

Results shown in Table 12 include performance comparison of the proposed branched ACC-PS model, branched ACC model, nonbranched ACC-PS, nonbranched ACC and several existing models reported from recent studies on energy expenditure prediction. As described above, these results indicate performance by experiment where sample size is equal to the total number of experiments from all subjects in all activity groups.

TABLE 13
Energy expenditure prediction by experiment

| | | | | | | | | 95% |
|---|---|---|---|---|---|---|---|---|
| | | | | | | | | interval of |
| | Branch | Sample | | | | | Bias, | agreement, |
| Model | Model | Size | RMSE_{MET} | ARD, % | AARD, % | RMSE % | METs | METs |
| ACC-PS | Sit | 22 | 0.2115 | 2.74 | 16.67 | 6.63 | −0.0059 | (−0.44, 0.43) |
| | Stand | 22 | 0.2484 | 4.16 | 20.30 | 7.79 | 0.000028 | (−0.51, 0.51) |
| | Walk | 73 | 0.7703 | 2.15 | 16.93 | 24.15 | −0.0321 | (−1.58, 1.52) |
| | Cycle | 22 | 0.8310 | 4.40 | 15.48 | 26.06 | 0.0921 | (−1.60, 1.78) |
| | Aggregated | 139 | 0.6616 | 2.92 | 17.19 | 20.75 | −0.0032 | (−1.33, 1.32) |
| ACC | Sit | 22 | 0.2115 | 2.74 | 16.67 | 6.63 | −0.0059 | (−0.44, 0.43) |
| | Stand | 22 | 0.2574 | 3.85 | 21.42 | 8.07 | −0.0108 | (−0.54, 0.52) |
| | Walk | 73 | 0.7648 | 2.02 | 16.75 | 23.98 | −0.0339 | (−1.57, 1.50) |
| | Cycle | 22 | 1.1634 | 7.19 | 23.81 | 36.48 | 0.0822 | (−2.29, 2.46) |
| | Aggregated | 139 | 0.7341 | 3.24 | 18.59 | 23.02 | −0.0075 | (−1.48, 1.47) |
| ACC-PS | | 139 | 0.9009 | 1.68 | 26.31 | 28.25 | −0.0208 | (−1.83, 1.79) |
| nonbranched | | | | | | | | |
| ACC nonbranched | | 139 | 0.9840 | 0.61 | 27.54 | 30.85 | −0.0221 | (−1.99, 1.95) |
| Studenmayer, 2009 [1] | | | 1.22 | — | — | — | — | — |
| Crouter, 2006 [2] | | | — | — | — | — | 0.1000 | (−1.4, 1.5) |
| Brage, 2007 [3]* | | | [0.87, | — | — | — | — | — |
| | | | 1.11] | | | | | |

*[min, max] interval for walking/running activities from Brage, 2007, using 5^{th}, 6^{th}, and 7^{th} calibration levels, originally given in J · kg⁻¹ · min⁻¹, converted to kcal · kg⁻¹ · hour⁻¹ (MET).

As shown in Table 13, both branched models (ACC-PS and ACC) outperform existing models in several performance assessment measures. Branched models also exhibit significantly better prediction performance than nonbranched models in all of the reported characteristics. The same improvement in performance is shown when comparing branched or nonbranched ACC-PS models to ACC models. At the same time, both nonbranched models achieve almost the same level of performance as the existing models found in the literature, (as indicated by similar levels of RMSE_{MET}, bias and 95% intervals of agreement).

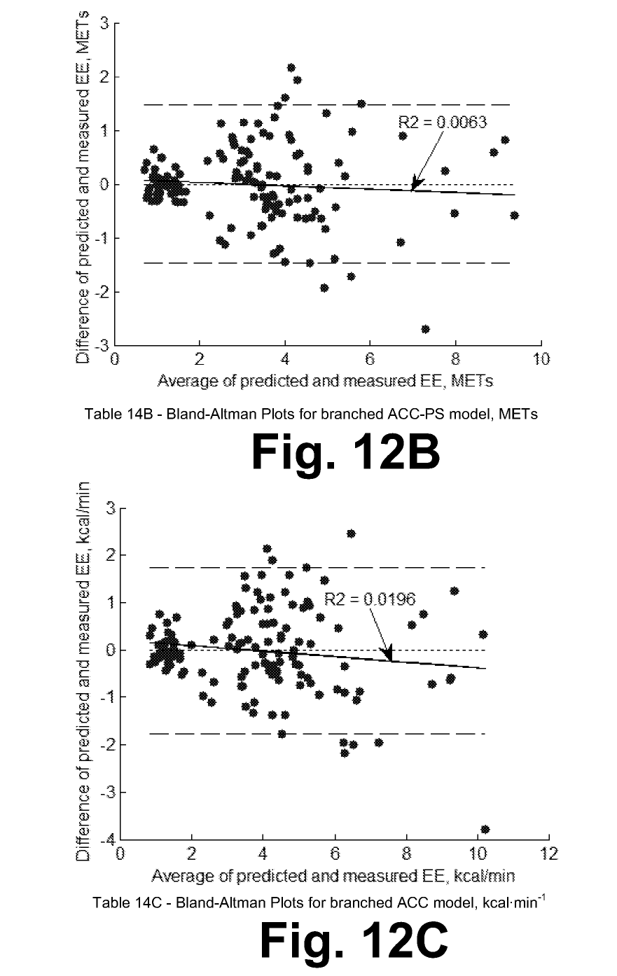

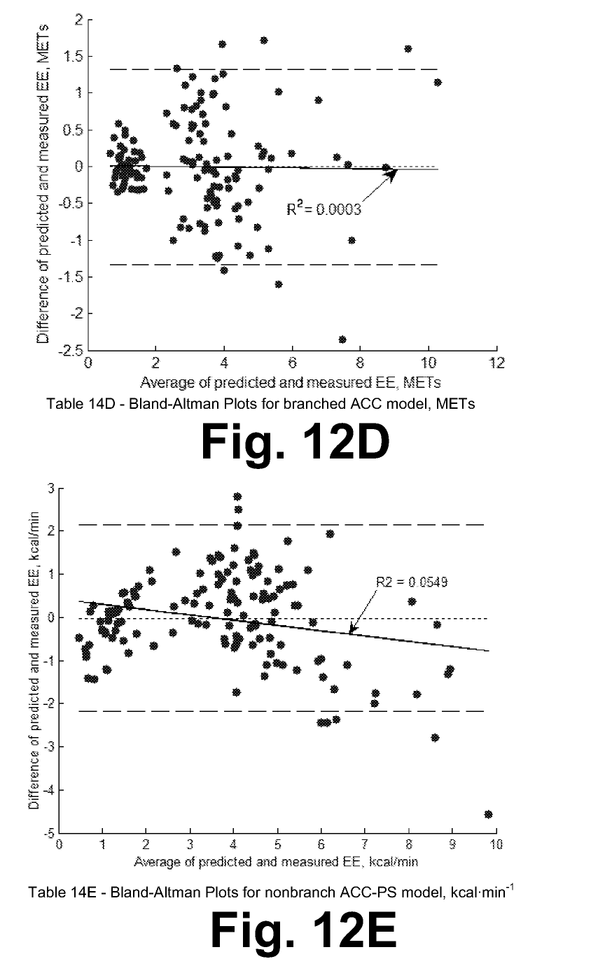

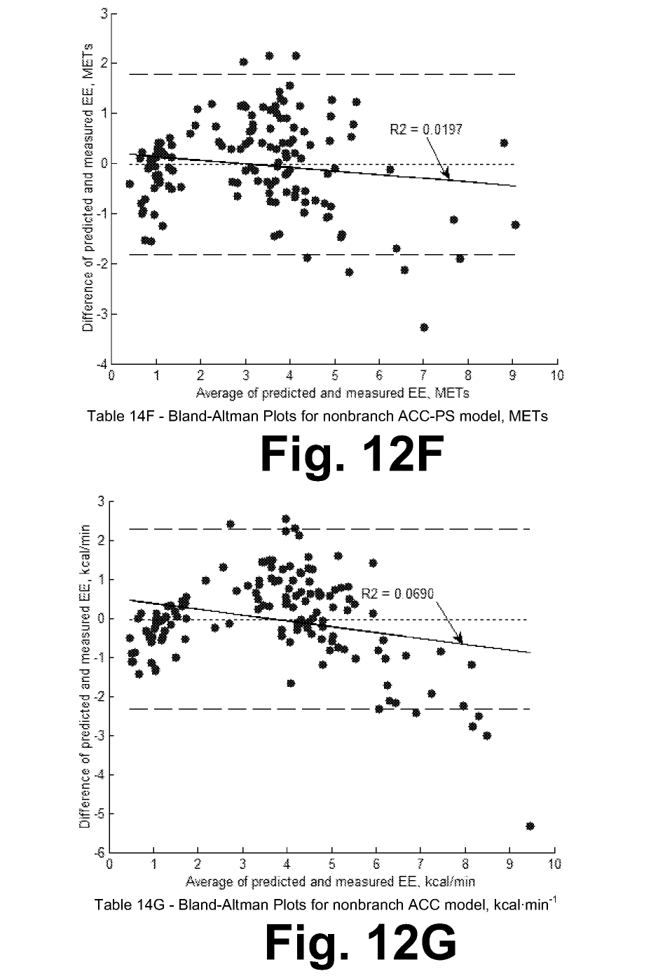

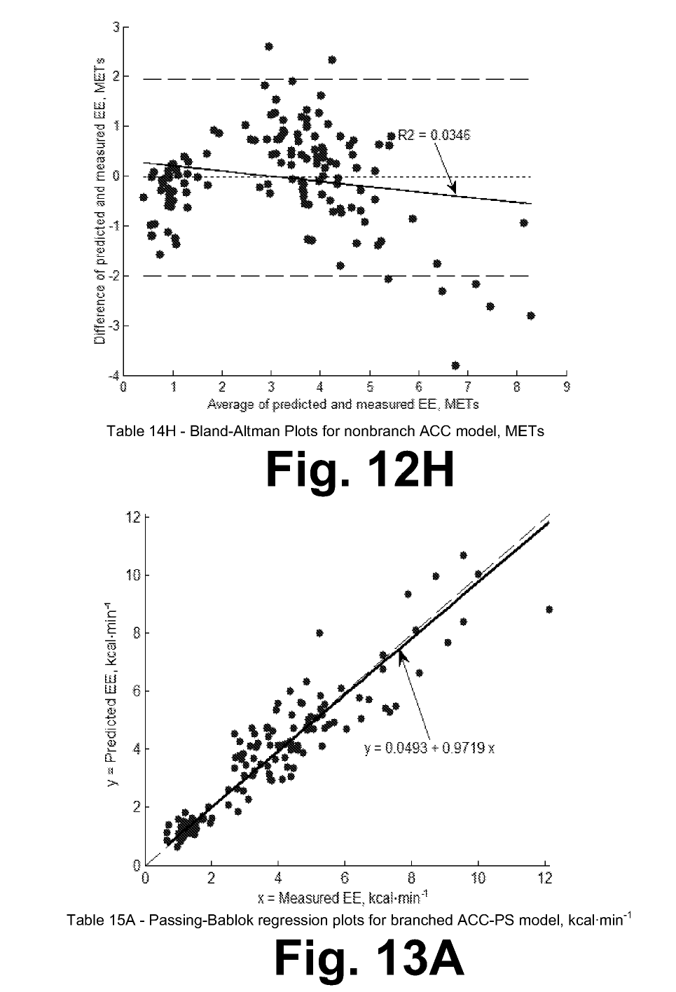

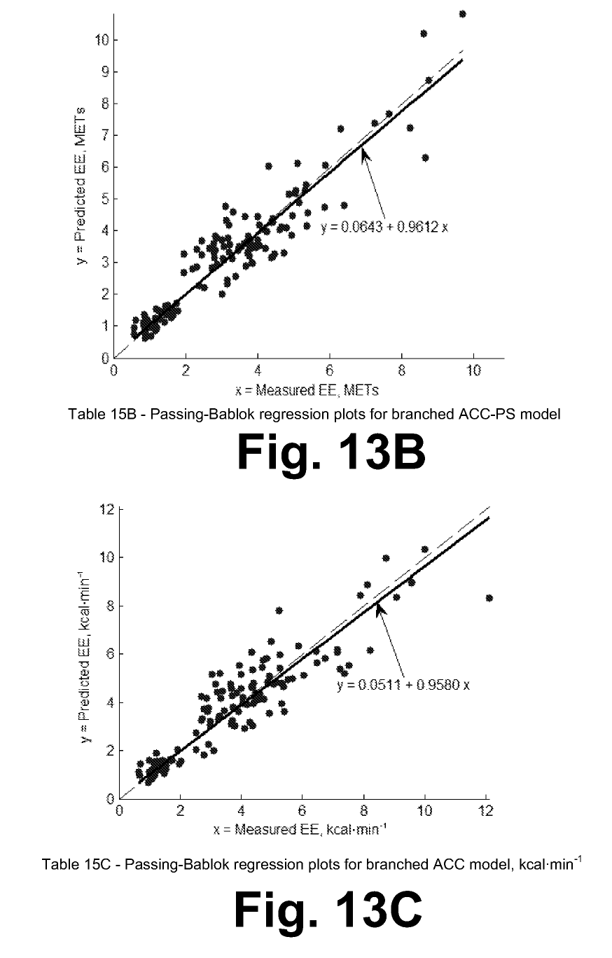

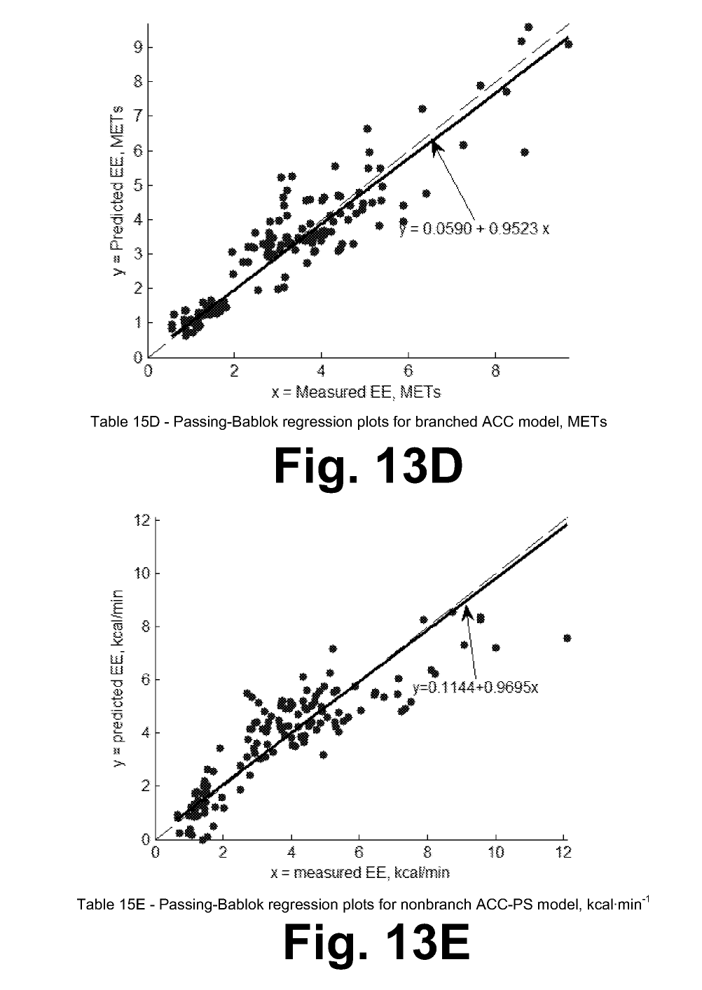

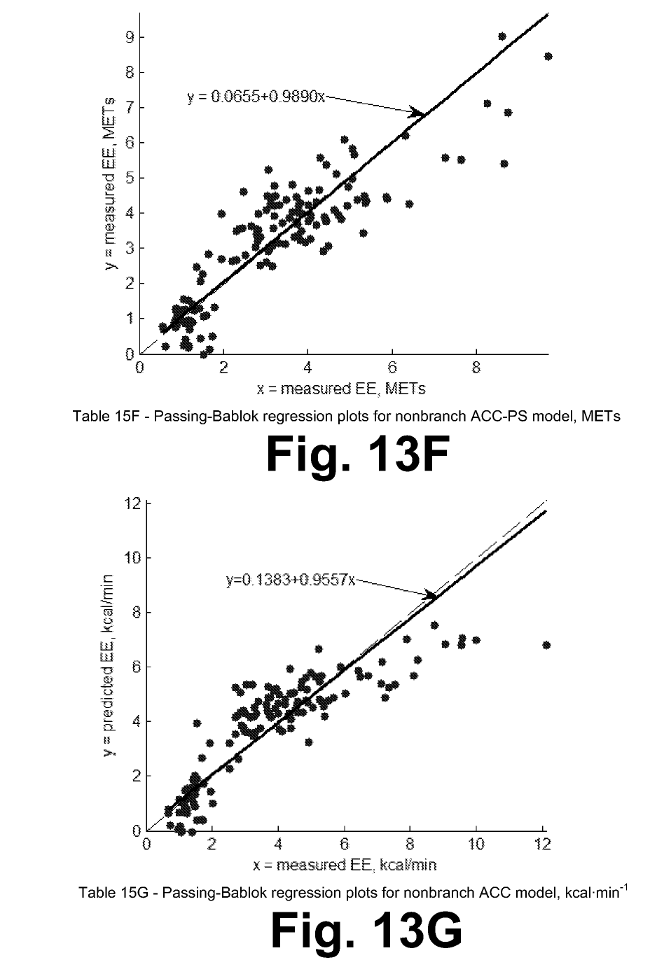

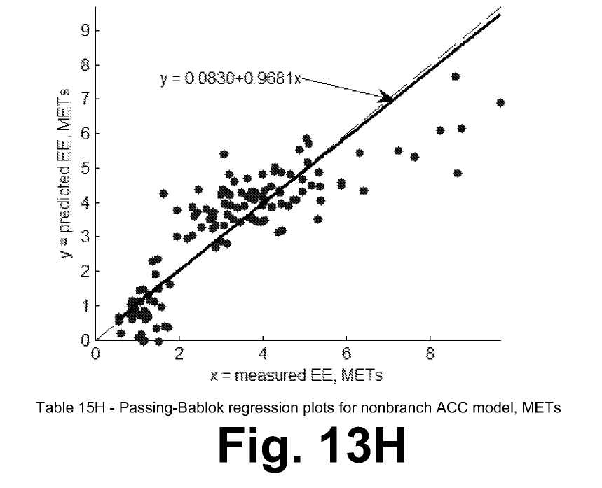

Bland-Altman plots (constructed for both EE, kcal·min⁻¹ and EE, METs prediction) for all four shoe-based models are shown in Tables 14A-14H in FIGS. 12A-12H. Tables 14A-14D are Bland-Altman plots for branched models and Tables 14E-14H are Bland-Altman plots for nonbranch models. The common characteristic for all these plots (models) is that the accuracy of prediction is slightly better for small than for large EE values. Visual comparison of the plots for branched and nonbranch models reveal that there is certainly a lot of unexplained variability in nonbranch models due to the fact that the plots show parabola-like patterns of the differences between predicted and measured EE. At the same time for branched models there is no pattern the differences between predicted and measured EE show over the range of the EE values. Improvement in the accuracy of prediction is also supported by the fact that the slopes of the fitted lines are significantly smaller for branched than for the nonbranch model as well as the fit is stronger for nonbranch models (greater R² values).

Passing-Bablok regression analysis for the four shoe-based models was conducted using Matlab. Results of this regression analysis are depicted in the plots of Tables 15A-15H as shown in FIGS. 15A-15H. Examination of the presence of fixed (intercept ≠0 if 95% CI does not contain 0) and proportional (slope ≠1 if 95% CI does not contain 1) bias of the models showed that none of the four models exhibited either kind of bias, as shown in Tables 15A-15H. All ACC-PS models (branched and nonbranched) provided better prediction over the ACC models as indicated by slope values closer to the unity than those of the ACC model. In addition, the branch models regression coefficients appeared to be more precise (as given by the narrower confidence intervals for both slope and intercept) than those for the “nonbranch” models. Test for linearity showed weak or absence of linearity for almost all nonbranched models while for branched models linearity was always very strong. Additional proof of the strength of linear relationship between predicted and measured EE values is given by correlation and concordance coefficients. There is clear tendency of both coefficients to increase from nonbranch to branch models and from ACC to ACC-PS models. Lack of linearity of the nonbranched models is also noticeable in their Passing-Bablok regression plots, which show clear curvature in the scatter plots unlike in those of the branched models.

Bias at mean, minimum and maximum measured EE values (as percentages of these values) was also evaluated using obtained Passing-Bablock regression equations (Tables 15A-15H). Both branched models (ACC-PS and ACC) showed significantly better accuracy of prediction than other reported models; bias at mean was 1.49% vs −5.84%. In addition, the ACC-PS model also showed improved results upon ACC model. (See Tables 16 and 17.) Nonbranched models revealed significant bias (10.48-16.52%) at the minimum measured EE values.

TABLE 16
Examination of the presence of fixed and proportional bias and linearity

| Model, | | 95% | | | 95% | Propor- | Test for | Corr. & |
|---|---|---|---|---|---|---|---|---|
| units of | Inter- | Cl for | Fixed | | Cl for | tional | linearity | concord |
| prediction | cept | intercept^{*} | bias? | Slope | slope^{*} | bias? | ^{*}p-value | coefficient |
| ACC-PS bran. | 0.0493 | (−0.1041, | No | 0.9719 | (0.9043, | No | Strong | 0.9304 |
| (kcal · min⁻¹) | | 0.2630) | | | 1.0313) | | linearity, | 0.9296 |
| | | | | | | | p-value | |
| | | | | | | | >0.1 | |
| ACC-PS bran. | 0.0643 | (−0.0950, | No | 0.9612 | (0.8953, | No | Strong | 0.9385 |
| (METs) | | 0.2338) | | | 1.0212) | | linearity, | 0.9385 |
| | | | | | | | p-value | |
| | | | | | | | >0.1 | |
| ACC bran. | 0.0511 | (−0.0914, | No | 0.9580 | (0.8970, | No | Strong | 0.9215 |
| (kcal · min⁻¹) | | 0.2266) | | | 1.0370) | | linearity, | 0.9201 |
| | | | | | | | p-value | |
| | | | | | | | >0.1 | |
| ACC bran. | 0.0590 | (−0.0661, | No | 0.9523 | (0.8870, | No | Strong | 0.9235 |
| (METs) | | 0.2188) | | | 1.0258) | | linearity, | 0.9230 |
| | | | | | | | p-value | |
| | | | | | | | >0.1 | |
| ACC-PS | 0.1144 | (−0.1864, | No | 0.9695 | (0.8726, | No | Weak | 0.8776 |
| nonbran. | | 0.3257) | | | 1.0804) | | linearity | 0.8717 |
| | | | | | | | 0.05< | |
| | | | | | | | p-value | |
| | | | | | | | <0.1 | |
| ACC-PS | 0.0655 | (−0.1696, | No | 0.9890 | (0.8911, | No | Strong | 0.8815 |
| nonbran. | | 0.2635) | | | 1.0883) | | linearity, | 0.8794 |
| (METs) | | | | | | | p-value | |
| | | | | | | | >0.1 | |
| ACC nonbran. | 0.1383 | (−0.1398, | No | 0.9557 | (0.8526, | No | No linearity | 0.8586 |
| (kcal · min⁻¹) | | 0.4876) | | | 1.0793) | | p-value | 0.8503 |
| | | | | | | | <0.01 | |
| ACC nonbran. | 0.0830 | (−0.1254, | No | 0.9681 | (0.8672, | No | No linearity | 0.8562 |
| (METs) | | 0.3343) | | | 1.088) | | p-value | 0.8521 |
| | | | | | | | <0.01 | |

*Computed as a result of Passing-Bablock regression estimation.

TABLE 17
Estimation of bias^{#}

| | Bias at | Bias at | Bias at |
|---|---|---|---|
| Model, units of prediction | mean, % | min, % | max, % |
| ACC-PS, kcal · min⁻¹ | 1.49 | −4.55 | 2.40 |
| ACC-PS, METs | 1.87 | −7.50 | 3.22 |
| ACC, kcal · min⁻¹ | −2.82 | 3.55 | −3.77 |
| ACC, METs | −2.92 | 5.65 | −4.16 |
| Nonbranched ACC-PS, | 0.03 | 14.29 | −2.10 |
| kcal · min⁻¹ | | | |
| Nonbranched ACC-PS, | 0.96 | 10.48 | −0.42 |
| METs | | | |
| Nonbranched ACC, | −0.72 | 16.52 | −3.29 |
| kcal · min⁻¹ | | | |
| Nonbranched ACC, METs | −0.59 | 11.48 | −2.33 |
| Thompson [4], kJ · min⁻¹ | −5.84 | −1.00 | −8.00 |

^{#}Bias is calculated from the Passing-Bablok regression line Ŷ = a + bX, where Y is the predicted value and X is the criterion. Reported bias values are computed as (Ŷ(Xᵢ) − Xᵢ)/Xᵢ for the criterion energy expenditure Xᵢ as mean, minimum, and maximum values observed in the sample.

The prediction of energy expenditure was estimated by subject as indicated in Table 18. Total energy expenditure (TEE) for each subject was computed as the sum of energy expenditures (in kcal·kg⁻¹) over all 1-min activities/experiments extrapolated over 780 min. proportionally to the original time allocated to each activity (2:2:6:2). The 780 min. was chosen as the length of a hypothetical 13-hour active wake cycle. The value of TEE was then normalized by an individual's weight. Initially, walking experiments included 7 activities (walk 1.5, walk 2.5, walk 3.5, jog 4.5, uphill, downhill, loaded). Four subjects had missing data for the jogging experiment, and, thus, the jogging was dropped in calculation of the TEE.

As can be seen from Table 18, the branched ACC-PS model performed slightly better than models constructed using accelerometer and heart rate, achieving 9.35% SEE versus 9.89%. The difference in the performance can be attributed to the difference in study protocols, in particular, different distribution of activities. Nevertheless, a branched ACC-PS model achieves accuracy in prediction similar to that of a branched model.

Although nonbranched models showed low SEEs, they provided biased estimates (as indicated by the greater deviation of the mean predicted TEE from the mean measured TEE) than the branched models.

TABLE 18
Energy expenditure prediction by subject

| | | ACC-PS | | No branch ACC- | No branch ACC |
|---|---|---|---|---|---|
| | | model | ACC model | PS model | model predicted |
| | TEE, | predicted TEE | predicted TEE | predicted TEE | TEE |
| Subject ID | (kcal · kg⁻¹) | (kcal · kg⁻¹) | (kcal · kg⁻¹) | (kcal · kg⁻¹) | (kcal · kg⁻¹) |
| 1 | 37.00 | 37.86 | 37.05 | 41.9229 | 41.7429 |
| 2 | 42.42 | 44.96 | 41.57 | 43.0567 | 42.8614 |
| 3 | 42.33 | 38.53 | 36.73 | 38.2194 | 39.6694 |
| 4 | 28.85 | 36.38 | 34.63 | 36.9798 | 37.1654 |
| 5 | 30.73 | 32.32 | 33.73 | 33.4540 | 34.2681 |
| 7 | 37.04 | 34.74 | 34.61 | 33.2850 | 33.1983 |
| 9 | 39.96 | 37.66 | 37.46 | 37.3655 | 38.7484 |
| 10 | 41.94 | 39.09 | 38.74 | 42.9712 | 42.7317 |
| 14 | 40.97 | 43.44 | 49.08 | 41.7202 | 44.0595 |
| 15 | 34.48 | 34.38 | 35.48 | 36.4614 | 36.0715 |
| 16 | 41.77 | 37.30 | 38.7 | 39.0697 | 40.4353 |
| Mean ± SD | 37.95 ± 4.81 | 37.88 ± 3.72 | 37.9786 ± 4.32 | 38.59 ± 3.52 | 39.17 ± 3.63 |
| (kcal · kg⁻¹) | | | | | |
| SEE | 0 | 3.55 | 4.16 | 3.80 | 3.55 |
| (kcal · kg⁻¹) | | | | | |
| SEE | 0 | 9.35 | 10.97 | 10.01 | 9.34 |
| (% of mean TEE) | | | | | |

G. Discussion

An earlier developed wearable shoe-based device with embedded accelerometer and pressure sensors was used for prediction of energy expenditure. The signals obtained from the shoe-based device was previously used to classify postures and activities into four groups: sit, stand, walk and cycle. A model was proposed that branched according to these actual performed groups of activities to be able to later combine the activity classification algorithm with this energy prediction model.

The proposed branched model that used both accelerometer signals and pressure sensors signals (branched ACC-PS) significantly improved the accuracy of prediction upon the branched model based solely on accelerometer readings (branched ACC) achieving root mean squared error (RMSE) of 0.66 METs vs 0.73 METs. In particular, the improvement was most significant due to the decrease in error rate in Stand and Cycle branch models. Both branched model outperformed existing methods based on accelerometry, heart rate and branching. Introduction of pressure sensors provided valuable information which also made a positive impact on the prediction of nonbranched ACC-PS versus nonbranched ACC models.

Comparison of branched ACC-PS and ACC models to their nonbranched versions suggested that branching considerably improves the prediction by lowering systematic bias, error rates and the width of the interval of prediction. In addition, the results of performance of the nonbranched models showed that they achieve accuracy comparable to that of the existing studies.

The quality of prediction provided by the model that uses the data from both shoes is not significantly different from the model that used single shoe data. Therefore, for all practical purposes, the use of single shoe embedded sensors is validated.

Although the embodiments have been described with respect to particular apparatuses, configurations, components, systems and methods of operation, it will be appreciated by those of ordinary skill in the art upon reading this disclosure that certain changes or modifications to the embodiments and/or their operations, as described herein, may be made without departing from the spirit or scope of the present disclosure. Accordingly, the proper scope of the present disclosure is defined by the appended claims. The various embodiments, operations, components and configurations disclosed herein are generally provided as examples rather than limiting in scope.

What is claimed is:

1. A footwear system for monitoring weight, posture allocation, physical activity classification, and energy expenditure calculation comprising an accelerometer configured to obtain acceleration data indicative of movement of a user's foot or leg;
a pressure sensing device mounted in an insole and configured to obtain pressure data indicative of pressure applied by a user's foot to the insole; and
a transmitter communicatively coupled to both the accelerometer and the pressure sensing device and configured to transmit the acceleration and pressure data to a first processing device configured process the acceleration data and the pressure data to distinguish a first posture from a second posture different from the first posture and process the acceleration data and the pressure data to distinguish a first movement-based activity from a second movement-based activity different from the first movement-based activity.

2. The footwear system of claim 1, wherein the pressure sensing device comprises a capacitive pressure sensor.

3. The footwear system of claim 1, wherein the capacitive pressure sensor comprises a first conductive layer including least one conducting plate and a user's foot functions as a second conductive layer when the user's foot is positioned on the insole.

4. The footwear system of claim 3, wherein a capacitance of the capacitive pressure sensor increases when more pressure is applied to the insole by the user's foot.

5. The footwear system of claim 3, wherein the insole includes an insulating top insole layer that is configured to separate the user's foot and the at least one conducting plate when the user is wearing the shoe.

6. The footwear system of claim 3, wherein the capacitive pressure sensor includes a first conducting plate connected in series to a second conducting plate.

7. The footwear system of claim 1, wherein the pressure sensing device comprises a force sensitive resistor.

8. The footwear system of claim 1, further comprising the first processing device, the first processing device communicatively coupled to the transmitter and configured to receive the acceleration data and the pressure data and derive a first energy expenditure value from the acceleration data and the pressure data.

9. The footwear system of claim 8, wherein the first processing device is further configured to derive a weight of a user based on the processed acceleration and pressure data.

10. The footwear system of claim 8, further comprising a second processing device communicatively coupled to the first processing device and configured to derive a second energy expenditure value, wherein the first and second processing devices are configured to transmit the first and second energy expenditure values to a server configured to compare the first and second energy expenditure values from the first and second processing devices.

11. The footwear system of claim 1, further comprising a physiological sensor configured to obtain biomedical data, wherein the first processing device is further configured to receive the biomedical data and derive a first energy expenditure value from a combination of the biomedical data, the acceleration data, and the pressure data.

12. The footwear system of claim 1, wherein the distinguishable first and second movement-based activities are selected from a group comprising walking, cycling, or running.

13. The footwear system of claim 1, wherein the distinguishable first and second postures are selected from a group comprising standing, sitting, or lying down.

14. The footwear system of claim 1, wherein the accelerometer is coupled to a circuit board separate from the pressure sensing device.

15. A method executed by a processing device for recognizing posture and activities using the processing device, comprising
receiving pressure data indicative of pressure applied by a user's foot to the insole;
receiving acceleration data from an accelerometer indicative of movement of a user's foot or leg; and
processing the pressure and acceleration data so as to distinguish a first posture from a second posture and to distinguish a first movement-based activity from a second movement-based activity.

16. The method of claim 15, further comprising deriving a first energy expenditure value from a weight of a user, the pressure data and the acceleration data.

17. The method of claim 16, wherein the weight of the user is calculated from the pressure data and the acceleration data.

18. The method of claim 15, further comprising calculating a weight of a user from the pressure data and the acceleration data.

19. The method of claim 15, further comprising generating an alert based on the derived first energy expenditure value.

20. The method of claim 15, further comprising
calculating a first energy expenditure value based on the first actual posture or motion-based activity of the user;
calculating a second energy expenditure value based a second actual posture or motion-based activity of the user;
combining the first energy expenditure value and the second energy expenditure value to obtain a total energy expenditure value; and
transmitting the total energy expenditure value to the user.

21. The method of claim 20, further comprising
comparing the total energy expenditure value to a threshold value; and
generating an alert if the total energy expenditure value is less than the threshold value.

22. A method executed by a processing device for deriving an energy expenditure value, comprising
obtaining pressure data using a capacitive pressure sensor indicative of pressure applied by a user's foot to the insole, the capacitive pressure sensor including one or more conducting plates;
obtaining acceleration data using an accelerometer indicative of movement of a user's foot or leg; and
transmitting the pressure and acceleration data to a processing device configured to process the pressure and acceleration data and derive an energy expenditure value based on the pressure and acceleration data.

23. The method of claim 22, wherein the capacitive pressure sensor includes a first conductive layer including a first conducting plate and a second conducting plate, and a foot of a user functions as a second conductive layer.

24. The method of claim 22, wherein the capacitive pressure sensor is provided in a sock.

25. The method of claim 22, wherein the capacitive pressure sensor is provided in an insole.

26. A computer-readable medium having computer-executable instructions for performing a computer process for recognizing posture and activities, the instructions comprising causing a computer processor device to
receive pressure data indicative of pressure applied by a user's foot to an insole;
receive acceleration data from an accelerometer indicative of movement of a user's foot or leg; and
process the pressure and acceleration data so as to distinguish a first posture from a second posture and to distinguish a first movement-based activity from a second movement-based activity.
